(19) United States
(12) Patent Application Publication
(10) Pub. No.: US 2008/0096848 A1
(43) Pub. Date: Apr. 24, 2008

(54) Substituted N-Aryl-9-Oxo-9H-Fluorene-1-Carboxamides and Analogs as Activators of Caspases and Inducers of Apoptosis

(75) Inventors: William E. Kemnitzer, San Diego, CA (US); Sui Xiong Cai, San Diego, CA (US); John A. Drewe, Carlsbad, CA (US); Nilantha Sudath Sirisoma, San Diego, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: CYTOVIA INC., SAN DIEGO CA (CA)

(21) Appl. No.: 11/664,060

(22) PCT Filed: Sep. 29, 2005
(86) PCT No.: PCT/US05/34890

§ 371 Date: Mar. 29, 2007

Related U.S. Application Data
(60) Provisional application No. 60/613,817, filed on Sep. 29, 2004.

(51) Int. Cl. A61K 31/16; A61P 35/00; A61K 31/655; C07C 245/22; A61K 31/5375; C07D 265/30; C07D 207/323; A61K 31/40; A61K 31/445; C07D 211/04; A61K 31/44; A61K 31/415; C07D 233/61; C07D 257/04; A61K 31/41; A61K 31/4164; C07D 233/00; C07C 233/01

(52) U.S. Cl. 514/150; 534/838; 514/237.8; 544/162; 548/564; 514/427; 514/315; 546/246; 546/329; 514/357; 514/399; 548/335.5; 548/254; 514/381; 514/406; 548/375.1; 564/123; 514/613

(57) ABSTRACT

The present invention is directed to substituted N-aryl-9-oxo-9H-fluorene-1-carboxamides and analogs thereof, represented by the general Formula I: (I) wherein R₁-R₈, X and Ar are defined herein. The present invention also relates to the discovery that compounds having Formula I are activators of caspases and inducers of apoptosis. Therefore, the activators of caspases and inducers of apoptosis of this invention can be used to induce cell death in a variety of clinical conditions in which uncontrolled growth and spread of abnormal cells occurs.

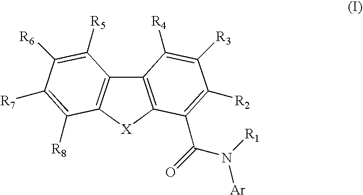

### BACKGROUND OF THE INVENTION

1. Field of the Invention

This invention is in the field of medicinal chemistry. In particular, the invention relates to substituted N-aryl-9-oxo-9H-fluorene-1-carboxamides and analogs, and the discovery that these compounds are activators of caspases and inducers of apoptosis. The invention also relates to the use of these compounds as therapeutically effective anti-cancer agents.

2. Description of Background Art

Organisms eliminate unwanted cells by a process variously known as regulated cell death, programmed cell death, or apoptosis. Such cell death occurs as a normal aspect of animal development, as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane-enclosed particles containing intracellular material) (Orrenius, S., J Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall, pp. 9-34 (1981)). A cell activates its internally-encoded suicide program as a result of either internal or external signals. The suicide program is executed through the activation of a carefully regulated genetic program (Wyllie, et al., Int. Rev. Cyt. 68:251 (1980); Ellis, et al., Ann. Rev. Cell Bio. 7:663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

It has been found that a group of proteases are a key element in apoptosis (see, e.g., Thornberry, Chemistry and Biology 5:R97—R103 (1998); Thornberry, British Med. Bull. 53:478-490 (1996)). Genetic studies in the nematode Caenorhabditis elegans revealed that apoptotic cell death involves at least 14 genes, 2 of which are the pro-apoptotic (death-promoting) ced (for cell death abnormal) genes, ced-3 and ced-4. CED-3 is homologous to interleukin 1 beta-converting enzyme, a cysteine protease, which is now called caspase 1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activates the caspase cascade. This makes the cancer cells lose their capacity to undergo cellular suicide so the cells become immortal—they become cancerous. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (Schmitt, et al., Biochem. Cell. Biol. 75:301-314 (1997)). BCL-like proteins include BCL-xL and BAX-alpha, which appear to function upstream of caspase activation. BCL-xL appears to prevent activation of the apoptotic protease cascade, whereas BAX-alpha accelerates activation of the apoptotic protease cascade.

It has been shown that chemotherapeutic (anti-cancer) drugs can trigger cancer cells to undergo suicide by activating the dormant caspase cascade. This may be a crucial aspect of the mode of action of most, if not all, known anticancer drugs (Los, et al., Blood 90(8):3118-3129 (1997); Friesen, et al., Nat. Med. 2:574 (1996)). The mechanism of action of current antineoplastic drugs frequently involves an attack at specific phases of the cell cycle. In brief, the cell cycle refers to the stages through which cells normally progress during their lifetime. Normally, cells exist in a resting phase termed Gₒ. During multiplication, cells progress to a stage in which DNA synthesis occurs, termed S. Later, cell division, or mitosis, occurs in a phase called M. Antineoplastic drugs, such as cytosine arabinoside, hydroxyurea, 6-mercaptopurine, and methotrexate are S phase specific, whereas antineoplastic drugs, such as vincristine, vinblastine, and paclitaxel are M phase specific. Many slow-growing tumors, e.g. colon cancers, exist primarily in the Gₒ phase, whereas rapidly proliferating normal tissues, e.g. bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, et al., eds., Goodman and Gilman's The Pharmacological Basis of Therapeutics, Ninth Edition, McGraw-Hill, New York, pp. 1225-1287 (1996)). Thus, it is clear that the possibility exists for the activation of the caspase cascade, although the exact mechanisms for doing so are not clear at this point. It is equally clear that insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could also involve the enhancement of the apoptotic process through the administration of appropriate caspase cascade activators and inducers of apoptosis.

### SUMMARY OF THE INVENTION

The present invention is related to the discovery that substituted N-aryl-9-oxo-9H-fluorene-1-carboxamides and analogs, as represented in Formulae I-IV, are activators of the caspase cascade and inducers of apoptosis. Therefore, the first aspect of the present invention is directed to the use of compounds of Formulae I-IV as inducers of apoptosis.

A second aspect of the present invention is to provide a method for treating, preventing or ameliorating neoplasia and cancer by administering a compound of Formulae I-IV to a mammal in need of such treatment.

A third aspect of the present invention is to provide novel compounds of Formulae I-IV, and to also provide for the use of these novel compounds for treating, preventing or ameliorating neoplasia and cancer.

A fourth aspect of the present invention is to provide a pharmaceutical composition useful for treating disorders responsive to the induction of apoptosis, containing an effective amount of a compound of Formulae I-IV in admixture with one or more pharmaceutically acceptable carriers or diluents.

A fifth aspect of the present invention is directed to methods for the preparation of novel compounds of Formulae I-IV.

### DETAILED DESCRIPTION OF THE INVENTION

The present invention arises out of the discovery that substituted N-aryl-9-oxo-9H-fluorene-1-carboxamides and analogs are potent and highly efficaceous activators of the caspase cascade and inducers of apoptosis. Therefore, these compounds are useful for treating disorders responsive to induction of apoptosis.

Specifically, compounds useful in this aspect of the present invention are substituted N-aryl-9-oxo-9H-fluorene-1-carboxamides and analogs as represented by Formula I: 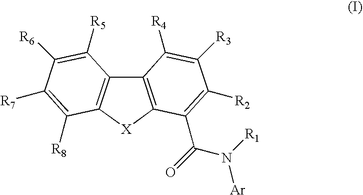
and pharmaceutically acceptable salts and prodrugs thereof, wherein:

X is CR₉R₁₀, O, NR₉, S, C═O, SO, or SO₂;

Ar is optionally substituted and is aryl, heteroaryl, saturated carbocyclic, partially saturated carbocylic, saturated heterocyclic, partially saturated heterocyclic, arylalkyl, or heteroarylalkyl;

R₁ is hydrogen or optionally substituted C₁₋₁₀ alkyl;

R₂-R₈ are independently hydrogen, halo, haloalkyl, aryl, optionally substituted fused aryl, optionally substituted fused heteroaryl, carbocyclic, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate; and

R₉ and R₁₀ are independently hydrogen, hydroxy or optionally substituted C₁₋₁₀ alkyl.

Preferred compounds falling within the scope of Formula I include compounds wherein R₂-R₈ are independently hydrogen, halo, haloalkyl, aryl, optionally substituted fused heteroaryl, carbocyclic, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate. Preferred compounds also include compounds wherein X is C═O. Preferred compounds also include compounds wherein X is CH₂.

One embodiment of the present invention is directed to compounds of Formula II: 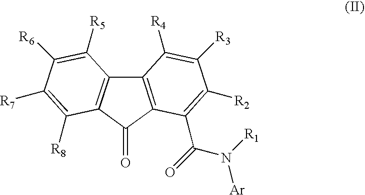
and pharmaceutically acceptable salts and prodrugs thereof, where R₁-R₈ and Ar are as defined above.

Preferred compounds falling within the scope of Formula II include compounds wherein R₂-R₈ are independently hydrogen, halo, haloalkyl, aryl, optionally substituted fused heteroaryl, carbocyclic, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate. Preferred compounds also include compounds wherein Ar is an optionally substituted phenyl or pyridyl.

Another embodiment of the present invention is directed to compounds of Formula III: 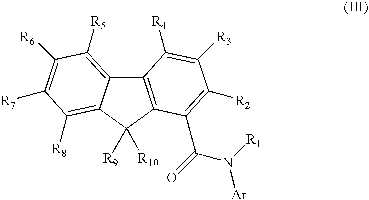
and pharmaceutically acceptable salts and prodrugs thereof, wherein:

R₁-R₁₀, and Ar are as defined above.

Another embodiment of the present invention is directed to compounds of Formula IV: 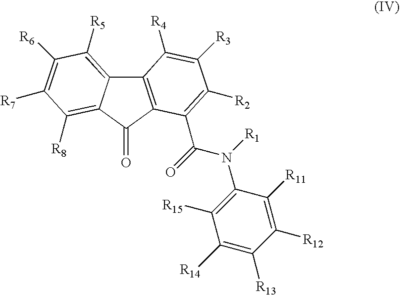
and pharmaceutically acceptable salts and prodrugs thereof, wherein:

R₁-R₈ are as described above; and

R₁₁-R₁₅ are independently hydrogen, halo, haloalkyl, aryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, nitro, amino, cyano, acylamido, hydroxy, thiol, acyloxy, azido, alkoxy, carboxy, carbonylamido, alkylthiol, alkylsulfonyl or alkylcarboxylate.

Preferably, R₁₁-R₁₅ are independently hydrogen, halo, C₁-C₆ haloalkyl, C₆-C₁₀ aryl, heteroaryl, heterocyclo, C₄-C₇ cycloalkyl, C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl)C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy, carboxy, (C₁-C₆)alkylsulfonyl or (C₁-C₆)alkylcarboxylate.

Preferred compounds falling within the scope of Formula IV include compounds wherein one of the R₁₁ or R₁₅ is not hydrogen.

Exemplary preferred compounds that may be employed in the method of invention include, without limitation:
N-(1-Naphthalen-1-yl)-9-oxo-9H-fluorene-1-carboxamide;
N-(2-Methylphenyl)-9-oxo-9H-fluorene-1-carboxamide;
N-(2-Phenylphenyl)-9-oxo-9H-fluorene-1-carboxamide;
N-(2-Difluoromethoxyphenyl)-9-oxo-9H-fluorene-1-carboxamide;
N-(2-(Methoxycarbonyl)phenyl)-9-oxo-9H-fluorene-1-carboxamide;
N-(2-Cholorophenyl)-9-oxo-9H-fluorene-1-carboxamide;
N-(2-Fluorophenyl)-9-oxo-9H-fluorene-1-carboxamide;
N-(2-Cyanophenyl)-9-oxo-9H-fluorene-1-carboxamide;
N-(2-Bromophenyl)-9-oxo-9H-fluorene-1-carboxamide;
N-(2-Ethoxyphenyl)-9-oxo-9H-fluorene-1-carboxamide;
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide;
N-(3-Methylpyridin-2-yl)-9-Oxo-9H-fluorene-1-carboxamide;
N-(2-(1H-Pyrazol-1-yl)-phenyl)-9H-fluorene-1-carboxamide;
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
N-(2-(1H-Pyrazol-1-yl)phenyl)-dibenzothiophene-1-carboxamide;
9-Hydroxy-N-(2-methylphenyl)-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide.

The present invention is also directed to novel compounds within the scope of Formulae I-IV. Exemplary preferred compounds that may be employed in this invention include, without limitation:
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide;
N-(3-Methylpyridin-2-yl)-9-oxo-9H-fluorene-1-carboxamide;
N-(2-(1H-Pyrazol-1-yl)-phenyl)-9H-fluorene-1-carboxamide;
N-(2-Bromomethylphenyl)-9-oxo-9H-fluorene-1-carboxamide;
N-(2-Methoxyphenyl)-9-oxo-9H-fluorene-1-carboxamide;
N-(2-(1H-pyrazol-1-yl)phenyl)-6,7,8,9-tetrahydro-5H-carbazole-1-carboxamide;
N-Methyl-N-(2-methylphenyl)-9-oxo-9H-fluorene-1-carboxamide;
N-(2-Methylcyclohexyl)-9-oxo-9H-fluorene-1-carboxamide;
N-(2-(1H-pyrazol-1-yl)phenyl)-2,3-dihydrobenzofuran-7-carboxamide;
N-(2-Dimethylaminomethylphenyl)-9-oxo-9H-fluorene-1-carboxamide;
N-(4-Methylpyridin-3-yl)-9-oxo-9H-fluorene-1-carboxamide;
N-(2-(4-Methylpiperazin-1-yl-methyl)phenyl)-9-oxo-9H-fluorene-1-carboxamide;
N-(2-(1H-pyrazol-1-yl)phenyl)-dibenzofuran-1-carboxamide;
N-(2-Nitrophenyl)-9-oxo-9H-fluorene-1-carboxamide;
N-(2-Amino-phenyl)-9-oxo-9H-fluorene-1-carboxamide;
N-(2-Azido-phenyl)-9-oxo-9H-fluorene-1-carboxamide;
N-Methyl-N-(2-(1H-pyrazol-1-yl)phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
N-(2-(1H-Pyrazol-1-yl)phenyl)-dibenzothiophene-1-carboxamide;
9,9-Dioxo-N-(2-(1H-pyrazol-1-yl)phenyl)-dibenzothiophene-1-carboxamide;
9-Oxo-N-(2-(1H-pyrazol-1-yl)phenyl)-dibenzothiophene-1-carboxamide;
N-(2-Hydroxy-phenyl)-9-oxo-9H-fluorene-1-carboxamide;
9-Hydroxy-N-(2-methylphenyl)-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide.

Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which can be optionally substituted.

Useful alkoxy groups include oxygen substituted by one of the C₁₋₁₀ alkyl groups mentioned above, which can be optionally substituted.

Useful alkylthio groups include sulphur substituted by one of the C₁₋₁₀ alkyl groups mentioned above, which can be optionally substituted. Also included are the sulfoxides and sulfones of such alkylthio groups.

Useful amino groups include —NH₂, —NHR₁₆, and —NR₁₆R₁₇, wherein R₁₆ and R₁₇ are C₁₋₁₀ alkyl or cycloalkyl groups, aryl or heteroaryl groups, or arylalkyl or heteroarylalkyl groups, or R₁₆ and R₁₇ are combined with the N to form a cycloamino structure, such as a piperidine, or R₁₆ and R₁₇ are combined with the N and other groups to form a cycloamino structure, such as a piperazine. The alkyl, cycloalkyl, aryl, heteroaryl, cycloamino groups can be optionally substituted.

Optional substituents on the alkyl groups include one or more halo, hydroxy, carboxyl, amino, nitro, cyano, C₁-C₆ acylamino, C₁-C₆ acyloxy, C₁-C₆ alkoxy, aryloxy, alkylthio, C₆-C₁₀ aryl, C₄-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated and unsaturated heterocyclic, or heteroaryl. Optional substituents on the aryl, aralkyl and heteroaryl groups include one or more halo, C₁-C₆ haloalkyl, optionally substituted C₆-C₁₀ aryl, optionally substituted heteroaryl, optionally substituted C₄-C₇ cycloalkyl, optionally substituted C₁-C₆ alkyl, C₂-C₆ alkenyl, C₂-C₆ alkynyl, C₆-C₁₀ aryl(C₁-C₆)alkyl, C₆-C₁₀ aryl(C₂-C₆)alkenyl, C₆-C₁₀ aryl(C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy, carboxy, (C₁-C₆)alkylsulfonyl and (C₁-C₆)alkylcarboxylate.

Useful aryl groups are C₆₋₁₄ aryl, especially C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as defined above, as well as cycloalkenyl groups, such as cyclopentenyl, cycloheptenyl and cyclooctenyl.

Useful halo or halogen groups include fluoro, chloro, bromo and iodo.

Useful arylalkyl groups include any of the above-mentioned C₁₋₁₀ alkyl groups substituted by any of the above-mentioned C₆₋₁₄ aryl groups. Useful values include benzyl, phenethyl and naphthylmethyl.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino groups are any C₁₋₆ acyl (alkanoyl) attached to an amino nitrogen, e.g., acetamido (acetylamino), propionamido, butanoylamido, pentanoylamido, hexanoylamido, as well as aryl-substituted C₂₋₆ substituted acyl groups.

Useful acyloxy groups are any C₁₋₆ acyl (alkanoyl) attached to an oxy (—O—) group, e.g., formyloxy, acetoxy, propionoyloxy, butanoyloxy, pentanoyloxy, hexanoyloxy and the like.

Useful saturated or partially saturated heterocyclic groups include tetrahydrofuranyl, pyranyl, piperidinyl, piperazinyl, 4-methyl-piperazinyl, 4-pyridyl-piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

Useful heteroaryl groups include any one of the following: thienyl, benzo[b]thienyl, naphtho[2,3-b]thienyl, thianthrenyl, furanyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]-pyrimidin-4-one, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g. a pyridyl N-oxide, pyrazinyl N-oxide, pyrimidinyl N-oxide and the like.

Certain of the compounds of the present invention may exist as stereoisomers including optical isomers. The invention includes all stereoisomers and both the racemic mixtures of such stereoisomers, as well as the individual enantiomers that may be separated according to methods that are well known to those of ordinary skill in the art.

Examples of pharmaceutically acceptable addition salts include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine.

Examples of prodrugs of the compounds of the invention include the simple esters of carboxylic acid containing compounds (e.g. those obtained by condensation with a C₁₋₄ alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

The compounds of this invention may be prepared using methods known to those skilled in the art, or the novel methods of this invention. Specifically, compounds with Formulae I-IV can be prepared as illustrated by exemplary reactions in Scheme 1. 9-Oxo-9H-fluorene-1-carboxylic acid was converted to 9-oxo-9H-fluorene-1-carbonyl chloride by reaction with oxalyl chloride in a solvent, such as CH₂Cl₂. Coupling of the 9-oxo-9H-fluorene-1-carbonyl chloride with a substituted aniline, such as 1-(2-aminophenyl)-pyrrole, in the presence of a base, such as NEt₃, and a solvent, such as CH₂Cl₂, produced the product N-(2-(1H-pyrrolo-1-yl)-phenyl)-9-oxo-9H-fluorene-1-carboxamide.

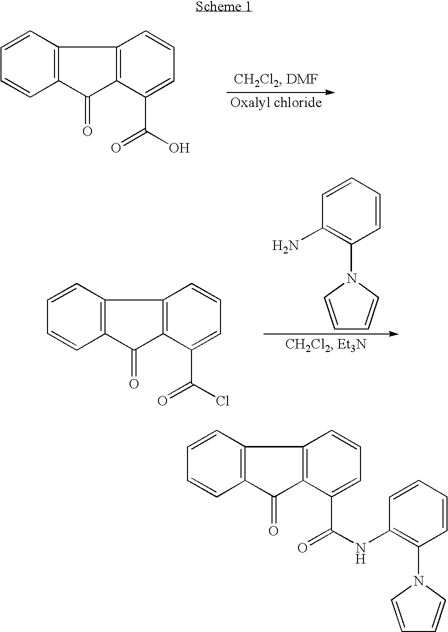

Alternatively, compounds with Formulae I-IV can be prepared as illustrated by exemplary reactions in Scheme 2. Coupling of the 9-oxo-9H-fluorene-1-carbonyl chloride with a substituted aniline, such as 2-(1H-pyrazol-1-yl)-aniline, in the presence of a base, such as NaH, and a solvent, such as THF, produced the product N-(2-(1H-pyrazol-1-yl)-phenyl)-9-oxo-9H-fluorene-1-carboxamide.

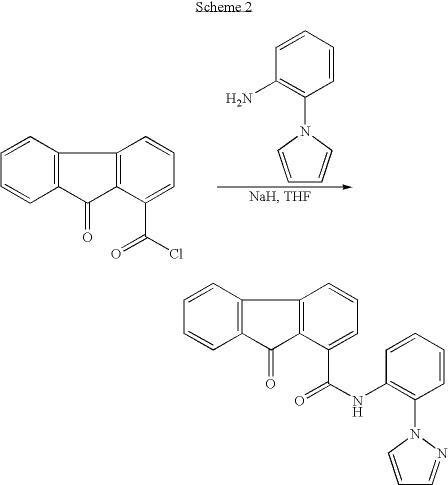

Compounds with Formulae I-IV can also be prepared as illustrated by exemplary reactions in Scheme 3-6.

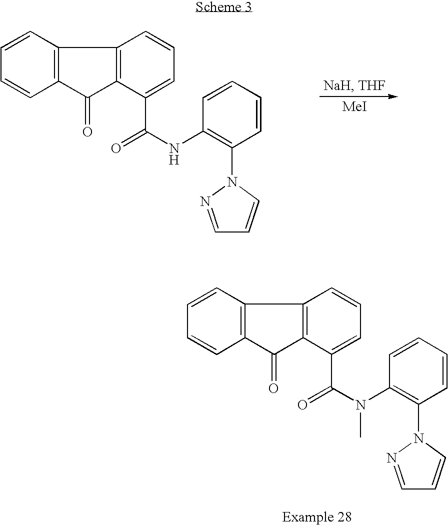

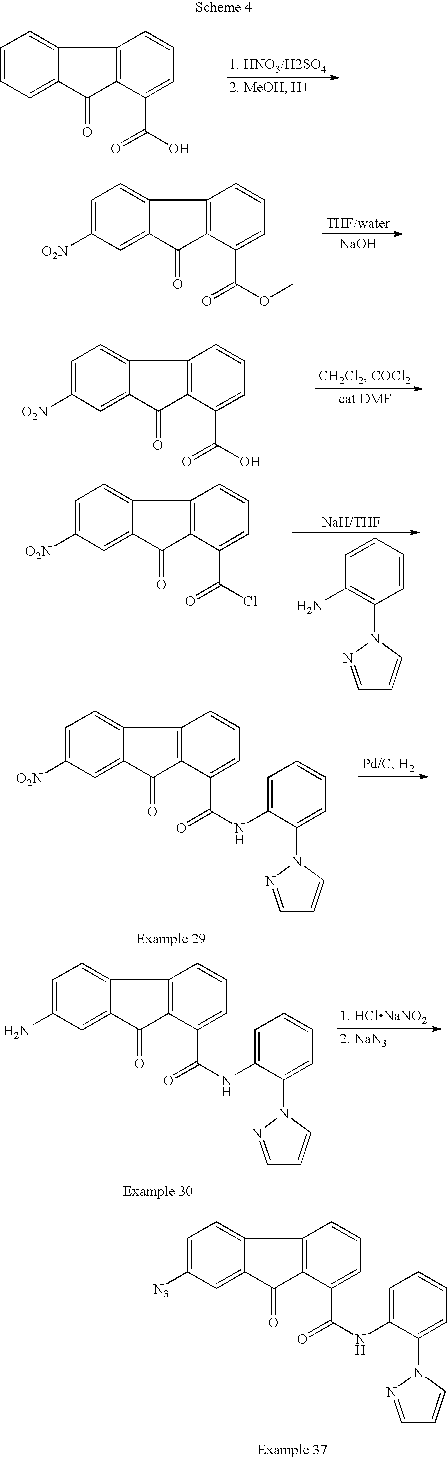

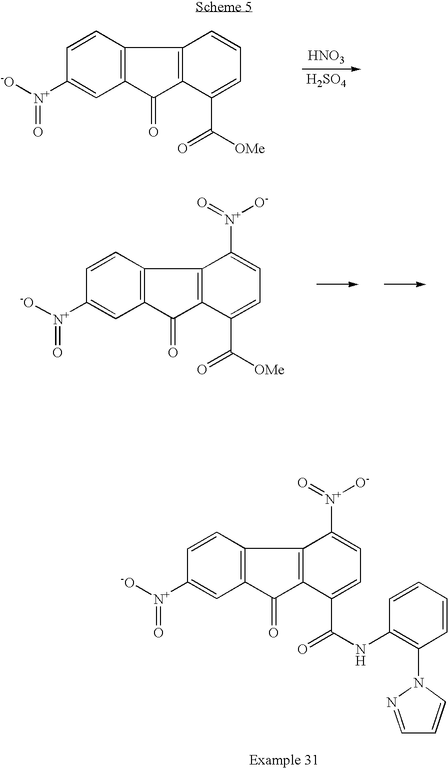

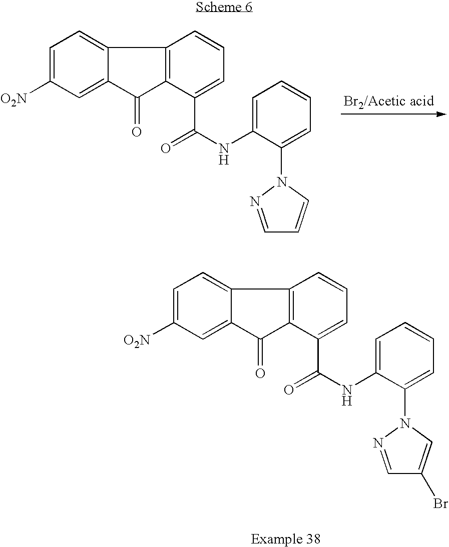

An important aspect of the present invention is the discovery that compounds having Formulae I-IV are activators of caspases and inducers of apoptosis. Therefore, these compounds are useful in a variety of clinical conditions in which there is uncontrolled cell growth and spread of abnormal cells, such as in the case of cancer.

Yet another important aspect of the present invention is the discovery that the compounds described herein are potent and highly efficacious activators of caspases and inducers of apoptosis in drug-resistant cancer cells, such as breast and prostate cancer cells, which enables these compounds to kill drug-resistant cancer cells. In comparison, most standard anti-cancer drugs are not effective in killing drug-resistant cancer cells under the same conditions. Therefore, compounds having Formulae I-IV are expected to be useful for the treatment of drug-resistant cancer in animals.

The present invention includes a therapeutic method useful to modulate in vivo apoptosis or in vivo neoplastic disease, comprising administering to a subject in need of such treatment an effective amount of a compound, or a pharmaceutically acceptable salt or prodrug of a compound described herein, which functions as a caspase cascade activator and inducer of apoptosis.

The present invention also includes a therapeutic method comprising administering to an animal an effective amount of a compound, or a pharmaceutically acceptable salt or prodrug of said compound of Formulae I-IV, wherein said therapeutic method is useful to treat cancer, which is a group of diseases characterized by the uncontrolled growth and spread of abnormal cells. Such diseases include, but are not limited to, Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyosarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

In practicing the therapeutic methods, effective amounts of compositions containing therapeutically effective concentrations of the compounds formulated for oral, intravenous, local and topical application (for the treatment of neoplastic diseases and other diseases in which caspase cascade mediated physiological responses are implicated), are administered to an individual exhibiting the symptoms of one or more of these disorders. The amounts are effective to ameliorate or eliminate one or more symptoms of the disorder. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In another embodiment, a pharmaceutical composition comprising a compound, or a pharmaceutically acceptable salt of a compound described herein, which functions as a caspase cascade activator and inducer of apoptosis in combination with a pharmaceutically acceptable vehicle, is provided.

Another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cancer chemotherapeutic agent, or a pharmaceutically acceptable salt of said agent. Examples of known anti-cancer agents which can be used for combination therapy include, but are not limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; and antibodies, such as Herceptin® and Rituxan®. Other known anti-cancer agents, which can be used for combination therapy, include arsenic trioxide, gamcitabine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In practicing the methods of the present invention, the compound of the invention may be administered together with the at least one known chemotherapeutic agent as part of a unitary pharmaceutical composition. Alternatively, the compound of the invention may be administered apart from the at least one known cancer chemotherapeutic agent. In this embodiment, the compound of the invention and the at least one known cancer chemotherapeutic agent are administered substantially simultaneously, i.e., the compounds are administered at the same time or one after the other, so long as the compounds reach therapeutic levels for a period of time in the blood.

It has been reported that alpha-1-adrenoceptor antagonists, such as doxazosin, terazosin, and tamsulosin, can inhibit the growth of prostate cancer cell via induction of apoptosis (Kyprianou, N., et al., Cancer Res. 60:4550-4555 (2000)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known alpha-1-adrenoceptor antagonists, or a pharmaceutically acceptable salt of said agent. Examples of known alpha-1-adrenoceptor antagonists, which can be used for combination therapy include, but are not limited to, doxazosin, terazosin, and tamsulosin.

It has been reported that sigma-2 receptors are expressed in high densities in a variety of tumor cell types (Vilner, B. J., et al., Cancer Res. 55: 408-413 (1995)) and that sigma-2 receptor agonists, such as CB-64D, CB-184 and haloperidol activate a novel apoptotic pathway and potentiate antineoplastic drugs in breast tumor cell lines (Kyprianou, N., et al., Cancer Res. 62:313-322 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known sigma-2 receptor agonists, or a pharmaceutically acceptable salt of said agent. Examples of known sigma-2 receptor agonists, which can be used for combination therapy include, but are not limited to, CB-64D, CB-184 and haloperidol.

It has been reported that combination therapy with lovastatin, a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HMG-CoA reductase inhibitor, or a pharmaceutically acceptable salt of said agent. Examples of known HMG-CoA reductase inhibitors, which can be used for combination therapy include, but are not limited to, lovastatin, simvastatin, pravastatin, fluvastatin, atorvastatin and cerivastatin.

It has been reported that HIV protease inhibitors, such as indinavir or saquinavir, have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HIV protease inhibitor, or a pharmaceutically acceptable salt of said agent. Examples of known HIV protease inhibitors, which can be used for combination therapy include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

It has been reported that synthetic retinoids, such as fenretinide (N-(4-hydroxyphenyl)retinamide, 4HPR), have good activity in combination with other chemotherapeutic agents, such as cisplatin, etoposide or paclitaxel in small-cell lung cancer cell lines (Kalemkerian, G. P., et al., Cancer Chemother. Pharmacol. 43:145-150 (1999)). 4HPR also was reported to have good activity in combination with gamma-radiation on bladder cancer cell lines (Zou, C., et al., Int. J. Oncol. 13:1037-1041 (1998)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known retinoid and synthetic retinoid, or a pharmaceutically acceptable salt of said agent. Examples of known retinoids and synthetic retinoids, which can be used for combination therapy include, but are not limited to, bexarotene, tretinoin, 13-cis-retinoic acid, 9-cis-retinoic acid, α-difluoromethylomithine, ILX23-7553, fenretinide, and N-4-carboxyphenyl retinamide.

It has been reported that proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known proteasome inhibitor, or a pharmaceutically acceptable salt of said agent. Examples of known proteasome inhibitors, which can be used for combination therapy include, but are not limited to, lactacystin, MG-132, and PS-341.

It has been reported that tyrosine kinase inhibitors, such as STI571 (Imatinib mesilate, Gleevec®), have potent synergetic effect in combination with other anti-leukemic agents, such as etoposide (Liu, W. M., et al., Br. J Cancer 86:1472-1478 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known tyrosine kinase inhibitor, or a pharmaceutically acceptable salt of said agent. Examples of known tyrosine kinase inhibitors, which can be used for combination therapy include, but are not limited to, Gleevec®, ZD1839 (Iressa), SH268, genistein, CEP2563, SU6668, SU11248, and EMD121974.

It has been reported that prenyl-protein transferase inhibitors, such as farnesyl protein transferase inhibitor R115777, possess preclinical antitumor activity against human breast cancer (Kelland, L. R., et. al., Clin. Cancer Res. 7:3544-3550 (2001)). Synergy of the protein farnesyltransferase inhibitor SCH66336 and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of said agent. Examples of known prenyl-protein transferase inhibitors, which can be used for combination therapy include, but are not limited to, R115777, SCH66336, L-778,123, BAL9611 and TAN-1813.

It has been reported that cyclin-dependent kinase (CDK) inhibitors, such as flavopiridol, have potent synergetic effect in combination with other anticancer agents, such as CPT-11, a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cyclin-dependent kinase inhibitor, or a pharmaceutically acceptable salt of said agent. Examples of known cyclin-dependent kinase inhibitor, which can be used for combination therapy include, but are not limited to, flavopiridol, UCN-01, roscovitine and olomoucine.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16 (4:3):17-21 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known COX-2 inhibitor, or a pharmaceutically acceptable salt of said agent. Examples of known COX-2 inhibitors, which can be used for combination therapy include, but are not limited to, celecoxib, valecoxib, and rofecoxib.

Another embodiment of the present invention is directed to a composition effective to inhibit neoplasia comprising a bioconjugate of a compound described herein, which functions as a caspase cascade activator and inducer of apoptosis, in bioconjugation with at least one known therapeutically useful antibody, such as Herceptin® or Rituxan®, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates could also enhance the anticancer effect of therapeutically useful antibodies, such as Herceptin® or Rituxan®.

Similarly, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a caspase cascade activator and inducer of apoptosis. The invention also relates to a method of treating cancer by surgically removing the cancer and then treating the animal with one of the pharmaceutical compositions described herein.

A wide range of immune mechanisms operate rapidly following exposure to an infectious agent. Depending on the type of infection, rapid clonal expansion of the T and B lymphocytes occurs to combat the infection. The elimination of the effector cells following an infection is one of the major mechanisms maintaining immune homeostasis. This deletion of reactive cells has been shown to be regulated by a phenomenon known as apoptosis. Autoimmune diseases have been lately identified as a consequence of deregulated cell death. In certain autoimmune diseases, the immune system directs its powerful cytotoxic effector mechanisms against specialized cells, such as oligodendrocytes in multiple sclerosis, the beta cells of the pancreas in diabetes mellitus, and thyrocytes in Hashimoto's thyroiditis (Ohsako, S., et al., Cell Death Differ. 6(1):13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly and generalized lymphadenopathy, hypergammaglobulinemia, and autoantibody formation (Infante, A. J., et al., J. Pediatr. 133(5):629-633 (1998) and Vaishnaw, A. K., et al, J. Clin. Invest. 103(3):355-363 (1999)). It was reported that overexpression of Bcl-2, which is a member of the Bcl-2 gene family of programmed cell death regulators with anti-apoptotic activity, in developing B cells of transgenic mice, in the presence of T cell dependent costimulatory signals, results in the generation of a modified B cell repertoire and in the production of pathogenic autoantibodies (Lopez-Hoyos, M., et al., Int. J. Mol. Med. 1(2):475-483 (1998)). Therefore, it is evident that many types of autoimmune disease are caused by defects of the apoptotic process and one treatment strategy would be to turn on apoptosis in the lymphocytes that are causing autoimmune disease (O'Reilly, L. A. and Strasser, A., Inflamm. Res. 48(1):5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al, J. Immunol. 162(1):603-608 (1999), reported that by direct injection of DNA expression vectors encoding FasL into the inflammed thyroid, the development of lymphocytic infiltration of the thyroid was inhibited and induction of the death of infiltrating T cells was observed. These results show that FasL expression on thyrocytes may have a curative effect on ongoing EAT by inducing death of pathogenic autoreactive infiltrating T lymphocytes.

Bisindolylmaleimide VII is known to potentiate Fas-mediated apoptosis in human astrocytoma 1321N1 cells and in Molt-4T cells, both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. Zhou, T., et al., Nat. Med. 5(1):42-8 (1999), reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore, the application of a Fas-dependent apoptosis enhancer, such as bisindolylmaleimide VIII, may be therapeutically useful for the more effective elimination of detrimental cells and inhibition of T cell-mediated autoimmune diseases. Therefore, an effective amount of a compound, or a pharmaceutically acceptable salt or prodrug of a compound described herein, which functions as a caspase cascade activator and inducer of apoptosis, should be an effective treatment for autoimmune disease.

Psoriasis is a chronic skin disease, which is characterized by scaly red patches. Psoralen plus ultraviolet A (PUVA) is a widely-used and effective treatment for psoriasis vulgaris. Coven, T. R., et al., Photodermatol. Photoimmunol. Photomed. 15(1):22-7 (1999), reported that lymphocytes treated with psoralen 8-MOP or TMP plus UVA displayed DNA degradation patterns typical of apoptotic cell death. Ozawa, M., et al, J. Exp. Med. 189(4):711-718 (1999), reported that induction of T cell apoptosis could be the main mechanism by which 312-nm UVB resolves psoriasis skin lesions. Low doses of methotrexate may be used to treat psoriasis to restore a clinically normal skin. Heenen, M., et al., Arch. Dermatol. Res. 290(5):240-245 (1998), reported that low doses of methotrexate may induce apoptosis and this mode of action could explain the reduction in epidermal hyperplasia during treatment of psoriasis with methotrexate. Therefore, an effective amount of a compound, or a pharmaceutically acceptable salt or prodrug of a compound described herein, which functions as a caspase cascade activator and inducer of apoptosis, should be an effective treatment for psoriasis.

Synovial cell hyperplasia is a characteristic of patients with rheumatoid arthritis (RA). Excessive proliferation of RA synovial cells that, in addition, are defective in synovial cell death might be responsible for the synovial cell hyperplasia. Wakisaka, S., et al., Clin. Exp. Immunol. 114(1):119-28 (1998), found that, although RA synovial cells could die via apoptosis through Fas/FasL pathway, apoptosis of synovial cells was inhibited by proinflammatory cytokines present within the synovium, and suggested that inhibition of apoptosis by the proinflammatory cytokines may contribute to the outgrowth of synovial cells and lead to pannus formation and the destruction of joints in patients with RA. Therefore, an effective amount of a compound, or a pharmaceutically acceptable salt or prodrug of a compound described herein, which functions as a caspase cascade activator and inducer of apoptosis, should be an effective treatment for rheumatoid arthritis.

There has been an accumulation of convincing evidence that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J Leukoc. Biol. 61(4):375-80 (1997)). Boirivant, M., et al., Gastroenterology 116(3):557-65 (1999), reported that lamina propria T cells isolated from areas of inflammation in Crohn's disease, ulcerative colitis, and other inflammatory states manifest decreased CD2 pathway-induced apoptosis, and that studies of cells from inflamed Crohn's disease tissue indicate that this defect is accompanied by elevated Bcl-2 levels. Therefore, an effective amount of a compound, or a pharmaceutically acceptable salt or prodrug of a compound described herein, which functions as a caspase cascade activator and inducer of apoptosis, should be an effective treatment for inflammation.

Caspase cascade activators and inducers of apoptosis may also be a desirable therapy in the elimination of pathogens, such as HIV, Hepatitis C and other viral pathogens. The long-lasting quiecence, followed by disease progression, may be explained by an anti-apoptotic mechanism of these pathogens leading to persistent cellular reservoirs of the virions. It has been reported that HIV-1 infected T leukemia cells or peripheral blood mononuclear cells (PBMCs) underwent enhanced viral replication in the presence of the caspase inhibitor Z-VAD-fink. Furthermore, Z-VAD-fink also stimulated endogenous virus production in activated PBMCs derived from HIV-1 infected asymptomatic individuals (Chinnaiyan, A., et al., Nat. Med. 3:333 (1997)). Therefore, apoptosis serves as a beneficial host mechanism to limit the spread of HIV and new therapeutics using caspase/apoptosis activators are useful to clear viral reservoirs from the infected individuals. Similarly, HCV infection also triggers anti-apoptotic mechanisms to evade the host's immune surveillance leading to viral persistence and hepatocarcinogenesis (Tai, D. I., et al., Hepatology 3:656-64 (2000)). Therefore, apoptosis inducers are useful as therapeutics for HIV and other infectious disease.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar, E., et al., Br. Med. Bull. 59:227-248 (2001)). Therefore, apoptosis inducers, which are antiproliferative, are useful as therapeutics for in-stent restenosis.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be orally administered to mammals, e.g. humans, at a dose of 0.0025 to 50 mg/kg, or an equivalent amount of the pharmaceutically acceptable salt thereof, per day of the body weight of the mammal being treated for apoptosis-mediated disorders. Preferably, about 0.01 to about 10 mg/kg is orally administered to treat or prevent such disorders. For intramuscular injection, the dose is generally about one-half of the oral dose. For example, a suitable intramuscular dose would be about 0.0025 to about 25 mg/kg, and most preferably, from about 0.01 to about 5 mg/kg. If a known cancer chemotherapeutic agent is also administered, it is administered in an amount which is effective to achieve its intended purpose. The amounts of such known cancer chemotherapeutic agents effective for cancer are well known to those of skill in the art.

The unit oral dose may comprise from about 0.01 to about 50 mg, preferably about 0.1 to about 10 mg of the compound of the invention. The unit dose may be administered one or more times daily as one or more tablets, each containing from about 0.1 to about 10, preferably about 0.25 to 50 mg of the compound or its solvates.

In a topical formulation, the compound may be present at a concentration of about 0.01 to 100 mg per gram of carrier.

In addition to administering the compound alone, the compounds of the invention may be administered as part of a pharmaceutical preparation containing suitable pharmaceutically acceptable carriers comprising excipients and auxiliaries, which facilitate processing of the compounds into preparations that can be used pharmaceutically. Preferably, the preparations, particularly those preparations, which can be administered orally and which can be used for the preferred type of administration, such as tablets, dragees, and capsules, and also preparations, which can be administered rectally, such as suppositories, as well as suitable solutions for administration by injection or orally, containing from about 0.01 to 99 percent, preferably from about 0.25 to 75 percent of active compound(s), together with the excipient.

Also included within the scope of the present invention are the non-toxic pharmaceutically acceptable salts of the compounds of the present invention. Acid addition salts are formed by mixing a solution of the particular apoptosis inducer of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the particular apoptosis inducer of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternative, or concurrent, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active compounds with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active compound doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active compounds in the form of granules, which may be mixed with fillers, such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active compounds with a suppository base. Suitable suppository bases are, e.g. natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils, e.g., sesame oil; or synthetic fatty acid esters, e.g., ethyl oleate or triglycerides or polyethylene glycol-400 (the compounds are soluble in PEG-400). Aqueous injection suspensions may contain substances, which increase the viscosity of the suspension include, e.g., sodium carboxymethyl cellulose, sorbitol, and/or dextran. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil such as almond oil, is admixed. A typical example of such a cream is one which includes about: 40 parts water, 20 parts beeswax, 40 parts mineral oil, and 1 part almond oil.

Ointments may be formulated by mixing a solution of the active ingredient in a vegetable oil, such as almond oil with warm soft paraffin and allowing the mixture to cool. A typical example of such an ointment is one which includes about: 30% almond oil and 70% white soft paraffin by weight.

The following examples are illustrative, but not limiting, of the method and compositions of the present invention. Other suitable modifications and adaptations of the variety of conditions and parameters normally encountered in clinical therapy, and which are obvious to those skilled in the art, are within the spirit and scope of the invention.

### EXAMPLE 1

### N-(2-Morpholinophenyl)-9-oxo-9H-fluorene-1-carboxamide

(a) 9-Oxo-9H-fluorene-1-carbonyl chloride. To a flame-dried reaction flask charged with a magnetic stir bar, under argon, at room temperature was added 9-Oxo-9H-fluorene-1-carboxylic acid (5.00 g, 22.3 mmol) and CH₂Cl₂ (55.0 mL). The brown suspension was cooled to 0° C. and then oxalyl chloride (2.0M in dichloromethane, 15.0 mL, 30.1 mmol) was added followed by anhydrous DMF (0.125 mL). The resulting brown solution was stirred at 0° C. for 1 h, equilibrated to room temperature, and then the solvent was evaporated to give the title compound as a brown solid (4.90 g, 90%): ¹H-NMR (DMSO-d₆): 7.93 (dd, J=7.6 and 1.0 Hz, 1H), 7.86 (d, J=7.4 Hz, 1H), 7.70-7.61 (m, 3H), 7.45-7.39 (m, 2H).

(b) N-(2-Morpholinophenyl)-9-oxo-9H-fluorene-1-carboxamide. To a flame-dried reaction flask charged with a magnetic stir bar, under argon, at room temperature was added 2-(4-morpholino)-aniline (0.147 g, 0.824 mmol), CH₂Cl₂ (4.2 mL) and Et₃N (0.115 mL, 0.824 mmol). The brown solution was cooled to 0° C. and then 9-oxo-9H-fluorene-1-carbonyl chloride (0.200 g, 0.824 mmol) was added in small portions over 2 min. The brown solution was stirred at 0° C. for 1 h, equilibrated to room temperature, and then the solvent was evaporated. The resulting brown oil was diluted with EtOAc (200 mL), washed with H₂O (2×40 mL), dried over Na₂SO₄, filtered and concentrated to yield a brown residue. Purification by flash column chromatography (silica gel, elution with EtOAc:Hexanes, 1:2) gave 0.152 g (48%) of the title compound as an orange solid: mp 154-156° C.; ¹H-NMR (CDCl₃): 11.23 (s, 1H), 8.45 (d, J=8.0 Hz, 1H), 8.13 (d, J=7.7 Hz, 1H), 7.72-7.54 (m, 5H), 7.38-7.33 (m, 1H), 7.25-7.13 (m, 3H), 3.79 (t, J=4.5 Hz, 4H), 2.98 (t, J=4.5 Hz, 4H).

### EXAMPLE 2

### N-(2-(1H-Pyrrolo-1-yl)-phenyl)-9-oxo-9H-fluorene-1-carboxamide

The title compound was prepared in a manner similar to Example 1b. From 1-(2-aminophenyl)-pyrrole (0.097 g, 0.62 mmol) and 9-oxo-9H-fluorene-1-carbonyl chloride (0.15 g, 0.62 mmol) was obtained 0.10 g (46%) of the title compound as a yellow solid: mp 202-203° C.; ¹H-NMR (CDCl₃): 11.11 (s, 1H), 8.21-8.14 (m, 2H), 7.69-7.59 (m, 3H), 7.54-7.52 (m, 2H), 7.45-7.28 (m, 4H), 6.91 (t, J=2.1 Hz, 2H), 6.23 (t, J=2.2 Hz, 2H).

### EXAMPLE 3

### 9-Oxo-N-(2-(piperidin-1-yl)-phenyl)-9H-fluorene-1-carboxamide

The title compound was prepared in a manner similar to Example 1b. From 2-piperidinoaniline (0.109 g, 0.618 mmol) and 9-oxo-9H-fluorene-1-carbonyl chloride (0.15 g, 0.62 mmol) was obtained 0.045 g (19%) of the title compound as a yellow solid: mp 152-154° C.; ¹H-NMR (CDCl₃): 10.69 (br s, 1H), 8.45 (d, J=8.0 Hz, 1H), 8.00 (d, J=7.7 Hz, 1H), 7.70-7.55 (m, 5H), 7.35 (t, J=7.0 Hz, 1H), 7.19-7.11 (m, 3H), 2.86 (t, J=4.9 Hz, 4H), 1.68-1.55 (m, 4H), 1.48-1.47 (m, 2H).

### EXAMPLE 4

### N-(2-(1H-Imidazol-1-yl)-phenyl)-9-oxo-9H-fluorene-1-carboxamide

The title compound was prepared in a manner similar to Example 1b. From 2-imidazol-1-yl-phenylamine (0.098 g, 0.62 mmol) and 9-oxo-9H-fluorene-1-carbonyl chloride (0.15 g, 0.62 mmol) was obtained 0.005 g (2%) of the title compound as a yellow solid: ¹H-NMR (CDCl₃): 11.64 (br s, 1H), 8.25 (d, J=7.4 Hz, 1H), 8.13 (d, J=8.5 Hz, 1H), 7.74 (s, 1H), 7.69-7.59 (m, 3H), 7.54-7.48 (m, 3H), 7.36-7.30 (m, 3H), 7.15 (d, J=16.2 Hz, 2H).

### EXAMPLE 5

### 9-Oxo-N-(pyridin-2-yl)-9H-fluorene-1-carboxamide

The title compound was prepared in a manner similar to Example 1b. From 2-aminopyridine (0.039 g, 0.41 mmol) and 9-oxo-9H-fluorene-1-carbonyl chloride (0.10 g, 0.41 mmol) was obtained 0.089 g (72%) of the title compound as a yellow solid: mp 164-165° C.; ¹H-NMR (CDCl₃): 12.74 (br s, 1H), 8.51 (d, J=4.9 Hz, 1H), 8.45 (d, J=8.2 Hz, 1H), 8.32 (dd, J=7.3 and 1.5 Hz, 1H), 7.78-7.69 (m, 2H), 7.67-7.61 (m, 2H), 7.54-7.52 (m, 2H), 7.35-7.29 (m, 1H), 7.11-7.07 (m, 1H).

### EXAMPLE 6

### 9-Oxo-N-phenyl-9H-fluorene-1-carboxamide

The title compound was prepared in a manner similar to Example 1b. From aniline (0.038 g, 0.41 mmol) and 9-oxo-9H-fluorene-1-carbonyl chloride (0.10 g, 0.41 mmol) was obtained 0.071 g (58%) of the title compound as a yellow solid: mp 183-185° C.; ¹H-NMR (CDCl₃): 12.41 (br s, 1H), 8.52-8.49 (m, 1H), 8.08-8.04 (m, 2H), 7.86-7.70 (m, 3H), 7.68-7.66 (m, 2H), 7.54-7.44 (m, 3H), 7.29-7.27 (m, 1H).

### EXAMPLE 7

### N-(2,6-Dimethylphenyl)-9-oxo-9H-fluorene-1-carboxamide

The title compound was prepared in a manner similar to Example 1b. From 2,6-dimethylaniline (0.125 g, 1.03 mmol) and 9-oxo-9H-fluorene-1-carbonyl chloride (0.250 g, 1.03 mmol) was obtained 0.311 g (92%) of the title compound as a yellow solid: ¹H-NMR (CDCl₃): 11.54 (br s, 1H), 8.37 (ddd, J=6.1, 1.4 and 0.8 Hz, 1H), 7.72-7.62 (m, 3H), 7.55 (dd, J=3.8 and 0.8 Hz, 2H), 7.36-7.31 (m, 1H), 7.14 (s, 3H), 2.32 (s, 6H).

### EXAMPLE 8

### N-(2-(1H-tetrazol-5-yl)phenyl)-9-oxo-9H-fluorene-1-carboxamide

The title compound was prepared in a manner similar to 1b. From 2-(1H-tetrazol-5-yl)-phenylamine (0.350 g, 2.17 mmol) and 9-oxo-9H-fluorene-1-carbonyl chloride (0.395 g, 1.63 mmol) was obtained 0.428 g (71%) of the title compound as a yellow solid: mp 253-257° C. (dec); ¹H-NMR (DMSO-d₆): 13.01 (s, 1H), 8.78 (d, J=8.4 Hz, 1H), 8.26 (d, J=7.7 Hz, 1H), 8.00 (d, J=7.3 Hz, 1H), 7.91 (d, J=7.3 Hz, 1H), 7.76 (t, J=7.7 Hz, 1H), 7.67 (t, J=7.3 Hz, 1H), 7.53 (t, J=6.1 Hz, 2H), 7.43-7.33 (m, 2H), 7.18 (t, J=7.7 Hz, 1H).

### EXAMPLE 9

### N-(2-(1H-Pyrazol-1-yl)-phenyl)-9-oxo-9H-fluorene-1-carboxamide

To a flame-dried 2-neck reaction flask charged with a magnetic stir bar, under argon, at room temperature was added NaH (60% mineral oil, 0.565 g, 14.1 mmol) and THF (47 mL). The gray suspension was cooled to 0° C. and then 2-(1H-pyrazol-1-yl)-aniline (1.50 g, 9.42 mmol) was added, forming a green suspension. The suspension was stirred for 20 min, and 9-oxo-9H-fluorene-1-carbonyl chloride (2.28 g, 9.42 mmol) was added in small portions over 2 min. The brown suspension was stirred at 0° C. for 1 h, equilibrated to room temperature, quenched with H₂O (5 mL) and then the solvent was evaporated. The resulting yellow residue was diluted with EtOAc (500 mL), washed with H₂O (3×40 mL), dried over MgSO₄, filtered and concentrated to yield a yellow solid. Recrystallization of the yellow solid with EtOH (220 mL) yielded 2.15 g (63%) of the title compound as a brown solid: mp 164-166° C.; ¹H-NMR (CDCl₃): 11.20 (br s, 1H), 8.50 (d, J=7.1 Hz, 1H), 7.86-7.83 (m, 2H), 7.66-7.64 (m, 1H), 7.61-7.52 (m, 5H), 7.46-7.40 (m, 2H), 7.34-7.29 (m, 2H), 6.41 (t, J=2.2 Hz, 1H).

### EXAMPLE 10

### N-(2-Dimethylaminophenyl)-9-oxo-9H-fluorene-1-carboxamide

(a) N¹,N¹-dimethylbenzene-1,2-diamine. To a hydrogenation reaction flask was added N,N-dimethyl-2-nitrobenzenamine (1.00 g, 6.02 mmol), EtOH (22 mL) and EtOAc (60 mL). To the resulting orange solution was added Pd/C (5%, 0.67 g) and then the black suspension was degassed three times and filled with H₂(g) (50 psi). The black suspension was shaken at room temperature for 5 h, filtered through celite (2 in h×1.5 in w), washed with additional EtOAc (50 mL) and concentrated to an orange residue. Purification by flash column chromatography (silica gel, with EtOAc:Hexanes, 1:15) gave 0.73 g (89%) of the title compound as an orange oil: ¹H-NMR (CDCl₃): 7.00 (dd, J=7.8 and 1.5 Hz, 1H), 6.90 (td, J=7.6 and 1.5 Hz, 1H), 6.76-6.68 (m, 2H), 3.93 (br s, 2H), 2.65 (s, 6H).

(b) N-(2-Dimethylaminophenyl)-9-oxo-9H-fluorene-1-carboxamide. The title compound was prepared in a manner similar to Example 9. From N¹,N¹-dimethybenzene-1,2-diamine (0.281 g, 2.06 mmol) and 9-oxo-9H-fluorene-1-carbonyl chloride (0.500 g, 2.06 mmol) was obtained 0.356 g (50%) of the title compound as a brown solid: mp 152-153° C.; ¹H-NMR (CDCl₃): 10.77 (br s, 1H), 8.42-8.40 (m, 1H), 8.06 (d, J=8.2 Hz, 1H), 7.72-7.54 (m, 5H), 7.36-7.33 (m, 1H), 7.18-7.12 (m, 3H), 2.70 (s, 6H).

### EXAMPLE 11

### N-(3-Methylpyridin-2-yl)-9-Oxo-9H-fluorene-1-carboxamide

The title compound was prepared in a manner similar to Example 9. From 2-amino-3-picoline (0.223 g, 2.06 mmol) and 9-oxo-9H-fluorene-1-carbonyl chloride (0.500 g, 2.06 mmol) was obtained 0.264 g (41%) of the title compound as a yellow solid: ¹H-NMR (CDCl₃): 12.21 (br s, 1H), 8.42-8.41 (m, 1H), 8.36 (dd, J=7.5 and 1.5 Hz, 1H), 7.73-7.55 (m, 5H), 7.38-7.34 (m, 2H), 7.17-7.13 (m, 1H), 2.41 (s, 3H).

### EXAMPLE 12

### N-(2-(1H-Pyrazol-1-yl)-phenyl)-9H-fluorene-1-carboxamide

(a) 9H-Fluorene-1-carbonyl chloride. The title compound was prepared in a manner similar to Example 1a. From fluorene-1-carboxylic acid (2.00 g, 9.51 mmol) and oxalyl chloride (2.0M in dichloromethane, 6.42 mL, 12.8 mmol) was obtained 1.65 g (76%) of the title compound as a brown solid: ¹H-NMR (DMSO-d₆): 8.17 (dd, J=7.7 and 1.1 Hz, 1H), 7.97 (d, J=6.3 Hz, 1H), 7.91 (dd, J=7.7 and 1.1 Hz), 7.65 (d, J=6.3 Hz, 1H), 7.54 (t, J=7.7 Hz, 1H), 7.42-7.36 (m, 2H), 4.22 (s, 2H).

(b) N-(2-(1H-pyrazol-1-yl)-phenyl)-9H-fluorene-1-carboxamide. The title compound was prepared in a manner similar to Example 9. From 2-(1H-pyrazol-1-yl)-aniline (0.500 g, 3.14 mmol) and 9H-fluorene-1-carbonyl chloride (0.718 g, 3.14 mmol) was obtained 0.502 g (45%) of the title compound as a brown solid: mp 179-180° C.; ¹H-NMR (CDCl₃): 11.17 (br s, 1H), 8.73 (d, J=8.2 Hz, 1H), 7.95 (d, J=7.4 Hz, 1H), 7.87 (d, J=2.5 Hz, 1H), 7.83-7.81 (m, 2H), 7.70 (d, J=7.7 Hz, 1H), 7.60-7.35 (m, 6H), 7.23-7.20 (m, 1H), 6.53-6.51 (m, 1H), 4.32 (s, 2H).

### EXAMPLE 13

### N-(2-Methylphenyl)-9H-fluorene-1-carboxamide

The title compound was prepared in a manner similar to example 12b. From 2-methylaniline and 9H-fluorene-1-carbonyl chloride was obtained the title compound as a solid.

### EXAMPLE 14

### N-(2-Bromomethylphenyl)-9-oxo-9H-fluorene-1-carboxamide

To an oven-dried reaction flask charged with a magnetic stir bar, under argon, at rt was added 9-oxo-N-(2-methylphenyl)-9H-fluorene-1-carboxamide (0.200 g, 0.638 mmol), N-bromosuccinimide (0.125 g, 0.702 mmol), AIBN (0.063 g, 0.38 mmol), bromine (0.036 mL, 0.70 mmol) and CCl₄ (6.4 mL). The orange suspension was heated to 100° C. while under a 254 nm UV-visible lamp. Once the reaction reached reflux, the UV-visible lamp was removed and the yellow suspension was heated at reflux for 3 h. The suspension was filtered through sintered glass and the solvent was evaporated to yield a yellow solid. Purification by flash column chromatography (silica gel, elution with EtOAc:Hexanes, 1:5) gave 0.003 g (1%) of the title compound as a yellow solid: ¹H-NMR (CDCl₃): 11.60 (s, 1H), 8.32 (d, J=7.8 Hz, 1H), 7.82-7.32 (m, 10H), 4.76 (s, 2H).

### EXAMPLE 15

### N-(2-Methoxyphenyl)-9-oxo-9H-fluorene-1-carboxamide

The title compound was prepared in a manner similar to example 9. From o-anisidine (0.500 g, 4.06 mmol) and 9-oxo-9H-fluorene-1-carbonyl chloride (0.985 g, 4.06 mmol) was obtained 0.505 g (38%) of the title compound as a yellow solid: ¹H-NMR (CDCl₃): 11.45 (br s, 1H), 8.48 (d, J=8.0 Hz, 1H), 8.26 (dd, J=7.7 and 1.4 Hz, 1H), 7.74-7.63 (m, 3H), 7.55-7.53 (m, 2H), 7.36-7.31 (m, 1H), 7.17-7.11 (m, 1H), 7.06-6.97 (m, 2H), 3.99 (s, 3H).

### EXAMPLE 16

### N-(2-(1H-pyrazol-1-yl)phenyl)-6,7,8,9-tetrahydro-5H-carbazole-1-carboxamide

(a) 6,7,8,9-Tetrahydro-5H-carbazole-1-carbonyl chloride. The title compound was prepared in a manner similar to example 1a. From 6,7,8,9-tetrahydro-5H-carbazole-1-carboxylic acid (1.00 g, 4.66 mmol), oxalyl chloride (2.0M in dichloromethane, 3.20 mL, 6.29 mmol) and DMF (0.025 mL) was obtained 0.500 g (46%) of the title compound as a brown solid: ¹H-NMR (DMSO-d₆): 10.60 (s, 1H), 7.65-7.59 (m, 2H), 7.05-7.00 (m, 1H), 2.76-2.74 (m, 2H), 2.64 (m, 2H), 1.81 (m, 4H).

(b) N-(2-(1H-pyrazol-1-yl)phenyl)-6,7,8,9-tetrahydro-5H-carbazole-1-carboxamide. The title compound was prepared in a manner similar to example 9. From 2-(1H-pyrazol-1-yl)-aniline (0.500 g, 3.14 mmol) and 6,7,8,9-tetrahydro-5H-carbazole-1-carbonyl chloride (0.733 g, 3.14 mmol) was obtained 0.180 g (16%) of the title compound as a white solid: ¹H-NMR (CDCl₃): 11.42 (br s, 1H), 9.93 (br s, 1H), 8.68 (d, J=7.1 Hz, 1H), 7.93 (d, J=1.9 Hz, 1H), 7.86 (d, J=2.5 Hz, 1H), 7.64 (d, J=7.4 Hz, 1H), 7.54 (d, J=7.7 Hz, 1H), 7.46-7.39 (m, 2H), 7.24-7.10 (m, 2H), 6.54-6.52 (m, 1H), 2.79-2.71 (m, 4H), 1.91-1.87 (m, 4H).

### EXAMPLE 17

### N-Methyl-N-(2-methylphenyl)-9-oxo-9H-fluorene-1-carboxamide

The title compound was prepared in a manner similar to example 9. From N-methyl-o-toluidine (0.099 g, 0.82 mmol) and 9-oxo-9H-fluorene-1-carbonyl chloride (0.200 g, 0.824 mmol) was obtained 0.099 g (36%) of the title compound as a yellow solid: ¹H-NMR (CDCl₃): 7.67-7.65 (m, 1H), 7.61-7.59 (m, 1H), 7.46-7.42 (m, 2H), 7.33-7.27 (m, 2H), 7.14-7.00 (m, 3H), 6.97-6.92 (m, 2H), 3.46 (s, 3H), 2.31 (s, 3H).

### EXAMPLE 18

### N-(2-Methylcyclohexyl)-9-oxo-9H-fluorene-1-carboxamide

The title compound was prepared in a manner similar to example 9. From 2-methylcyclohexylamine (0.093 g, 0.82 mmol) and 9-oxo-9H-fluorene-1-carbonyl chloride (0.200 g, 0.824 mmol) was obtained 0.131 g (50%) of the title compound as a brown solid: ¹H-NMR (CDCl₃): 9.92 (br s, 1H), 8.31-8.28 (m, 1H), 7.67-7.52 (m, 5H), 7.34-7.31 (m, 1H), 3.78-3.76 (m, 1H), 1.83-1.58 (m, 4H), 1.44-1.23 (m, 5H), 1.03-1.00 (m, 3H).

### EXAMPLE 19

### N-(2-(1H-pyrazol-1-yl)phenyl)-2,3-dihydrobenzofuran-7-carboxamide

(a) 2,3-Dihydrobenzofuran-7-carbonyl chloride. The title compound was prepared in a manner similar to example 1a. From 2,3-dihydrobenzofuran-7-carboxylic acid (1.00 g, 6.09 mmol), oxalyl chloride (2.0M in dichloromethane, 4.10 mL, 8.22 mmol) and DMF (0.040 mL) was obtained 0.50 g (46%) of the title compound as a yellow solid: ¹H-NMR (CDCl₃): 7.89-7.86 (m, 1H), 7.48-7.45 (m, 1H), 6.97-6.92 (m, 1H), 4.78 (t, J=8.7 Hz, 2H), 3.28 (t, J=8.7 Hz, 2H).

(b) N-(2-(1H-pyrazol-1-yl)phenyl)-2,3-dihydrobenzofuran-7-carboxamide. The title compound was prepared in a manner similar to example 9. From 2-(1H-pyrazol-1-yl)aniline (0.174 g, 1.09 mmol) and 2,3-dihydrobenzofuran-7-carbonyl chloride (0.200 g, 1.09 mmol) was obtained 0.123 g (37%) of the title compound as a brown solid: ¹H-NMR (CDCl₃): 10.25 (br s, 1H), 8.68 (dd, J=8.2 and 1.4 Hz, 1H), 7.92 (dd, J=8.0 and 0.8 Hz, 1H), 7.82 (d, J=1.9 Hz, 1H), 7.70 (d, J=2.2 Hz, 1H), 7.49-7.43 (m, 1H), 7.32 (t, J=1.5 Hz, 1H), 7.30 (d, J=1.4 Hz, 1H), 7.18 (td, J=7.6 and 1.4 Hz, 1H), 6.95 (t, J=7.7 Hz, 1H), 6.51 (t, J=2.1 Hz, 1H), 4.59 (t, J=8.8 Hz, 2H), 3.23 (t, J=8.7 Hz, 2H).

### EXAMPLE 20

### N-(2-Dimethylaminomethylphenyl)-9-oxo-9H-fluorene-1-carboxamide

(a) N,N-Dimethyl-(2-nitrophenyl)methenamine. To an oven-dried 1-neck 200 mL reaction flask charged with a magnetic stir bar, under argon, fitted with a reflux condenser at rt was added 1-(bromomethyl)-2-nitrobenzene (2.00 g, 9.26 mmol), dimethylamine (40% by weight, 5.93 mL, 47.2 mmol) and ethanol (46 mL). The yellow solution was refluxed at 105° C. for 1.5 hrs. The yellow solution was then cooled to rt and stirred under argon overnight. The reaction mixture was acidified using concentrated HCl (pH=1) and then the suspension was concentrated by rotary evaporation. The residue was then basified using 1M NaOH (pH=10) and then extracted with ether (2×125 mL). The combined organic extracts were washed with brine (2×30 mL), dried over MgSO₄, filtered and concentrated to yield an orange oil. Purification by flash column chromatography (silica gel, elution with EtOAc:Hexanes, 1:4) gave 0.875 g (52%) of the title compound as a yellow oil: ¹H-NMR (CDCl₃): 7.81 (d, J=8.0 Hz, 1H), 7.62-7.52 (m, 2H), 7.41-7.36 (m, 1H), 3.70 (s, 2H), 2.21 (s, 6H).

(b) 2-Dimethylaminomethyl-phenylamine. To a hydrogenation reaction flask was added N,N-dimethyl(2-nitrophenyl)methenamine (0.875 g, 4.86 mmol), EtOH (18 mL) and EtOAc (48 mL). To the resulting yellow solution was added Pd/C (5%, 0.59 g) and then the black suspension was degassed three times and filled with H_{2(g)} (50 psi). The black suspension was shaken at rt for 6 h, filtered through celite (2 in h×1.5 in w), washed with additional EtOAc (150 mL) and concentrated to an orange residue. Purification by flash column chromatography (silica gel, gradient with EtOAc:Hexanes, 1:5 to 1:2) gave 0.62 g (85%) of the title compound as an orange oil: ¹H-NMR (CDCl₃): 7.08 (td, J=7.6 and 1.6 Hz, 1H), 6.98-6.95 (m, 1H), 6.68-6.62 (2H), 4.47 (br s, 2H), 3.40 (s, 2H), 2.19 (s, 6H).

(c) N-(2-Dimethylaminomethylphenyl)-9-oxo-9H-fluorene-1-carboxamide. The title compound was prepared in a manner similar to example 9. From 2-dimethylaminomethyl-phenylamine (0.250 g, 1.66 mmol) and 9-oxo-9H-fluorene-1-carbonyl chloride (0.404 g, 1.66 mmol) was obtained 0.095 g (16%) of the title compound as a yellow solid: ¹H-NMR (CDCl₃): 11.41 (br s, 1H), 8.45 (d, J=8.0 Hz, 1H), 7.68-7.50 (m, 6H), 7.39-7.31 (m, 2H), 7.14-7.04 (m, 2H), 3.58 (s, 2H), 2.05 (s, 6H).

### EXAMPLE 21

### N-(4-Methylpyridin-3-yl)-9-oxo-9H-fluorene-1-carboxamide

The title compound was prepared in a manner similar to example 9. From 3-amino-4-methylpyridine (0.223 g, 2.06 mmol) and 9-oxo-9H-fluorene-1-carbonyl chloride (0.500 g, 2.06 mmol) was obtained 0.618 g (96%) of an orange solid: ¹H-NMR (CDCl₃): 11.90 (br s, 1H), 9.00 (s, 1H), 8.40-8.36 (m, 2H), 7.75-7.66 (m, 3H), 7.59-7.57 (m, 2H), 7.39-7.33 (m, 1H), 7.22 (d, J=4.4 Hz, 1H), 2.47 (s, 3H).

### EXAMPLE 22

### N-(2-(4-Methylpiperazin-1-yl-methyl)phenyl)-9-oxo-9H-fluorene-1-carboxamide

(a) 1-(2-Nitrobenzyl)-4-methylpiperazine. To an oven-dried 1-neck 50 mL reaction flask charged with a magnetic stir bar, under argon, fitted with a reflux condenser at rt was added 1-(bromomethyl)-2-nitrobenzene (0.400 g, 1.85 mmol) and anhydrous THF (9.3 mL). To the yellow solution was added 1-methyl-piperazine (0.226 mL, 2.04 mmol) and Et₃N (0.284 mL, 2.04 mmol). The resulting white suspension was refluxed at 82° C. for 3 h. The yellow solution was then cooled to rt and stirred under argon overnight. The reaction mixture was acidified using concentrated HCl (pH=1) and then the suspension was concentrated by rotary evaporation. The residue was then basified using 1M NaOH (pH=10) and then extracted with ether (2×75 mL). The combined organic extracts were washed with brine (2×15 mL), dried over MgSO₄, filtered and concentrated to yield an orange oil. Purification by flash column chromatography (silica gel, gradient elution with EtOAc, 100%; MeOH:EtOAc, 1:10; MeOH:EtOAc, 1:1) gave 0.276 g (64%) of the title compound as a yellow solid: ¹H-NMR (CDCl₃): 7.82-7.79 (m, 1H), 7.58-7.50 (m, 2H), 7.42-7.37 (m, 1H), 3.80 (s, 2H), 2.49 (br s, 8H), 2.30 (s, 3H).

(b) 2-(4-Methylpiperazin-1-yl-methyl)benzamine. The title compound was prepared in a manner similar to example 20b. From 1-(2-nitrobenzyl)-4-methyl piperazine (0.275 g, 1.17 mmol) and Pd/C (5%, 0.184 g) under H_{2(g)} (50 psi) was obtained 0.192 g (80%) of the title compound as an oil. ¹H-NMR (CDCl₃): 7.10-6.97 (m, 2H), 6.68-6.61 (m, 2H), 5.44 (br s, 2H), 3.51 (s, 2H), 2.47 (br s, 8H), 2.30 (s, 3H).

(c) N-(2-(4-Methylpiperazin-1-yl-methyl)phenyl)-9-oxo-9H-fluorene-1-carbox-amide. The title compound was prepared in a manner similar to example 9. From 2-((4-methylpiperazin-1-yl)-methyl)benzamine (0.180 g, 877 mmol) and 9-oxo-9H-fluorene-1-carbonyl chloride (0.213 g, 0.877 mmol) was obtained 0.028 g (8%) of the title compound as a yellow solid: ¹H-NMR (CDCl₃): 11.50 (br s, 1H), 8.48 (d, J=8.2 Hz, 1H), 7.68-7.51 (m, 6H), 7.39-7.31 (m, 2H), 7.14-7.05 (m, 2H), 3.64 (s, 2H), 2.32 (br s, 8H), 1.92 (s, 3H).

### EXAMPLE 23

### N-(2-(1H-pyrazol-1-yl)phenyl)-dibenzofuran-1-carboxamide

(a) Dibenzofuran-1-carboxylic acid. To an oven-dried 1-neck 100 mL reaction flask charged with a magnetic stir bar, under argon, at rt was added dibenzofuran (2.00 g, 11.9 mmol) and anhydrous THF (9.9 mL). The clear solution was cooled to −75° C. using a dry ice and acetone bath, then nBuLi (1.6M in hexanes, 7.43 mL, 11.9 mmol) was added dropwise over a period of 5 minutes. The yellow suspension was warmed to rt and stirred for 3 h. Using a dry ice and acetone bath, the suspension was cooled back down to −74° C. and added to a slurry of crushed CO₂ (150 mL) and anhydrous ether (100 mL). After 1 h, all of the CO₂ had evaporated and the suspension was diluted with ether (150 mL) and water (50 mL). The aqueous layer was extracted, acidified with concentrated HCl (pH=2) and then extracted with EtOAc (2×250 mL). The combined organic extracts were washed with brine (50 mL), dried over Na₂SO₄, filtered and concentrated to yield 2.31 g (92%) of the title compound as a yellow solid: ¹H-NMR (DMSO-d₆): 11.43 (s, 1H), 8.44-8.41 (m, 1H), 8.23-8.21 (m, 1H), 8.06-8.03 (m, 1H), 7.82-7.80 (m, 1H), 7.61-7.43 (m, 3H).

(b) Dibenzofuran-1-carbonyl chloride. The title compound was prepared in a manner similar to example 1a. From dibenzofuran-1-carboxylic acid (2.00 g, 9.43 mmol), oxalyl chloride (2.0M in dichloromethane, 6.36 mL, 12.7 mmol) and DMF (0.075 mL) was obtained 1.56 g (72%) of the title compound as a yellow solid: ¹H-NMR (DMSO-d₆): 8.44-8.42 (m, 1H), 8.24-8.21 (m, 1H), 8.06-8.03 (m, 1H), 7.83-7.80 (m, 1H), 7.62-7.43 (m, 3H).

(c) N-(2-(1H-pyrazol-1-yl)phenyl)-dibenzofuran-1-carboxamide. The title compound was prepared in a manner similar to example 9. From 2-(1H-pyrazol-1-yl)-aniline (0.300 g, 1.88 mmol) and dibenzofuran-4-carbonyl chloride (0.435 g, 1.88 mmol) was obtained 0.330 g (50%) of the title compound as a white solid: ¹H-NMR (DMSO-d₆): 11.16 (br s, 1H), 8.53 (d, J=8.0 Hz, 1H), 8.43-8.39 (m, 2H), 8.26 (d, J=7.4 Hz, 1H), 8.09 (d, J=7.7 Hz, 1H), 7.81 (d, J=1.9 Hz, 1H), 7.71-7.48 (m, 6H), 7.37 (t, J=7.7 Hz, 1H), 6.65 (t, J=2.0 Hz, 1H).

### EXAMPLE 24

### N-(2-Dimethylaminomethyl-phenyl)-9-oxo-9H-fluorene-1-carboxamide Hydrochloride

To an oven-dried 1-neck, 25 mL round bottom reaction flask charged with a magnetic stir bar, under argon, at rt was added N-(2-dimethylaminomethyl-phenyl)-9-oxo-9H-fluorene-1-carboxamide (0.309 g, 0.867 mmol) and anhydrous ether (2.2 mL). To the yellow suspension was added anhydrous 1,4-dioxane (2.0 mL) until all of the suspension had dissolved. Once a solution had formed, 4.0N HCl (in 1,4-dioxane, 0.66 ml) was added dropwise resulting in a yellow precipitate. The yellow precipitate was filtered, collected, and washed with additional anhydrous ether (15 mL). The yellow precipitate was dried under vacuo overnight to give 0.282 g (82%) of the title compound as a white solid: ¹H-NMR (DMSO-d₆): 10.58 (s, 1H), 10.51 (br s, 1H), 7.97 (d, J=7.4 Hz, 1H), 7.90 (d, J=7.7 Hz, 1H), 7.78-7.75 (m, 2H), 7.71-7.67 (m, 2H), 7.60-7.56 (m, 3H), 7.47-7.42 (m, 2H), 4.49 (d, J=5.5 Hz, 2H), 2.78 (d, J=4.2 Hz, 6H).

### EXAMPLE 25

### N-(2-Nitrophenyl)-9-oxo-9H-fluorene-1-carboxamide

The title compound was prepared in a manner similar to example 9. From 2-nitroaniline (1.00 g, 7.24 mmol) and 9-oxo-9H-fluorene-1-carbonyl chloride (1.76 g, 7.24 mmol) was obtained 0.420 g (17%) of the title compound as a yellow solid: ¹H-NMR (CDCl₃): 11.72 (s, 1H), 8.56 (dd, J=8.5 and 1.4 Hz, 1H), 8.17 (dd, J=8.2 and 1.4 Hz, 1H), 7.94 (dd, J=7.7 and 1.1 Hz, 1H), 7.74-7.67 (m, 3H), 7.64-7.62 (m, 1H), 7.58-7.56 (m, 2H), 7.38-7.30 (m, 2H).

### EXAMPLE 26

### N-(2-Amino-phenyl)-9-oxo-9H-fluorene-1-carboxamide

The title compound was prepared in a manner similar to example 20b. From N-(2-nitrophenyl)-9-oxo-9H-fluorene-1-carboxamide (0.408 g, 1.18 mmol) and Pd/C (5%, 0.275 g, 0.67 equiv. by weight) under H_{2(g)} (40 psi) was obtained 0.028 g (7%) of the title compound as a yellow solid: ¹H-NMR (CDCl₃): 11.54 (s, 1H), 8.31 (dd, J=7.3 and 1.8 Hz, 1H), 7.72-7.63 (m, 3H), 7.57-7.48 (m, 3H), 7.37-7.31 (m, 1H), 7.14-7.08 (m, 1H), 6.88-6.84 (m, 2H), 4.21 (br s, 2H).

### EXAMPLE 27

### N-(2-Azido-phenyl)-9-oxo-9H-fluorene-1-carboxamide

To an oven-dried 1-neck 100 mL reaction flask charged with a magnetic stir bar, under argon, at rt was added N-(2-Amino-phenyl)-9-oxo-9H-fluorene-1-carboxamid (0.025 g, 0.080 mmol), 1N HCl (1.3 mL), MeOH (0.75 mL) and concentrated HCl (0.050 mL). The resulting orange suspension was cooled in an ice bath to 0° C. and then a solution of NaNO₂ (0.026 g, 0.38 mmol, 4.7 equiv.) and H₂O (0.21 mL) was added over 10 minutes. The orange suspension was stirred at 0° C. for 0.5 h and then a solution of sodium azide (0.026 g, 0.40 mmol, 5.0 equiv.) and H₂O (0.300 mL) was added over 5 minutes. The yellow mixture was stirred at 0° C. for 1 h and then additional sodium azide (0.033 g, 0.51 mmol) was added in one portion. The yellow mixture was equilibrated to rt, stirred for 24 h, and then diluted with EtOAc (50 mL). The organic layer was then washed with NaHCO₃ (2×15 mL), brine (10 mL), dried over Na₂SO₄, filtered and concentrated to an orange oil. Purification by flash column chromatography (silica gel, elution with EtOAc:Hexanes, 1:4) gave 0.012 g (44%) of the title compound as a yellow solid: IR (KBr): 2120 cm⁻¹ (N₃-stretch); ¹H-NMR (CDCl₃): 11.54 (s, 1H), 8.28-8.25 (m, 2H), 7.76-7.65 (m, 3H), 7.57-7.56 (m, 2H), 7.37-7.33 (m, 2H), 7.26-7.23 (m, 2H).

### EXAMPLE 28

### N-Methyl-N-(2-(1H-pyrazol-1-yl)phenyl)-9-oxo-9H-fluorene-1-carboxamide

To an oven-dried 1-neck 25 mL reaction flask charged with a magnetic stir bar, under argon, at rt was added NaH (60% mineral oil, 0.008 g, 0.3 mmol) and THF (1.3 mL). The gray suspension was cooled to 0° C. and then N-(2-(1H-pyrazol-1-yl)phenyl)-9-oxo-9H-fluorene-1-carboxamide (0.096 g, 0.26 mmol) was added forming a brown suspension. After 5 minutes, methyl iodide (0.018 mL, 0.29 mmol) was added dropwise to the reaction suspension. The brown suspension was stirred at 0° C. for 1 h, equilibrated to rt, stirred overnight, and then the solvent was evaporated. The resulting residue was diluted with EtOAc (100 mL), washed with H₂O (2×20 mL), brine (20 mL), dried over MgSO₄, filtered and concentrated to yield a brown residue. Purification by flash column chromatography (silica gel, elution with EtOAc:Hexanes, 1:2) gave 0.013 g (13%) of the title compound as a yellow solid: ¹H-NMR (CDCl₃): 7.80-7.52 (m, 5H), 7.45-7.31 (m, 4H), 7.24-7.06 (m, 4H), 6.54-6.48 (m, 1H), 3.42 (d, J=1.5 Hz, 3H).

### EXAMPLE 29

### 7-Nitro-N-(2-(1H-pyrazol-1-yl)phenyl)-9-oxo-9H-fluorene-1-carboxamide

(a) 7-Nitro-9-oxo-9H-fluorene-1-carboxylic acid. To an oven-dried 1-neck 100 mL reaction flask charged with a magnetic stir bar, under argon, at rt was added 9-fluorenone-1-carboxylic acid (2.00 g, 8.92 mmol) and H₂SO₄ (8.9 mL). The brown suspension was cooled to 0° C. and then HNO₃ (0.41 mL, 9.8 mmol) were added over 1 minute. The brown suspension was equilibrated to rt and stirred overnight. The brown suspension was poured onto ice (100 mL) and a precipitate formed. The suspension was made alkaline by the addition of NaOH until the pH=14. The resulting green precipitate was filtered, washed with H₂O and hexanes and collected on a buchner funnel. Recrystallization of the green solid with EtOH (40 mL) yielded 0.62 g (26%) of the title compound as a yellow solid: ¹H-NMR (CDCl₃): 8.54 (dd, J=8.2 and 2.2 Hz, 1H), 8.26 (d, J=1.7 Hz, 1H), 8.18-8.14 (m, 2H), 7.79 (t, J=7.6 Hz, 1H), 7.59 (7.4 Hz, 1H).

(b) 7-Nitro-9-oxo-9H-fluorene-1-carbonyl chloride. The title compound was prepared in a manner similar to example 1a. From 7-nitro-9-oxo-9H-fluorene-1-carboxylic acid (0.500 g, 1.86 mmol), oxalyl chloride (2.0M in dichloromethane, 1.25 mL, 2.51 mmol) and DMF (0.025 mL) was obtained 0.511 g (95%) of the title compound as a yellow solid: ¹H-NMR (CDCl₃): 8.54 (d, J=1.7 Hz, 1H), 8.48 (dd, J=8.2 and 2.2 Hz, 1H), 7.90-7.88 (m, 1H), 7.80-7.75 (m, 3H).

(c) N-(2-(1H-pyrazol-1-yl)phenyl)-7-nitro-9-oxo-9H-fluorene-1-carboxamide. The title compound was prepared in a manner similar to example 9. From 2-(1H-pyrazol-1-yl)-aniline (0.138 g, 0.869 mmol) and 7-nitro-9-oxo-9H-fluorene-1-carbonyl chloride (0.250 g, 0.869 mmol) was obtained 0.242 g (68%) of the title compound as a yellow solid: ¹H-NMR (CDCl₃): 11.08 (s, 1H), 8.61 (d, J=8.2 Hz, 1H), 8.46 (dd, J=8.2 and 2.2 Hz, 1H), 8.39 (s, 1H), 7.90-7.86 (m, 2H), 7.81-7.70 (m, 3H), 7.56 (s, 1H), 7.50-7.42 (m, 2H), 7.30-7.27 (m, 1H), 6.46 (s, 1H).

### EXAMPLE 30

### 7-Amino-N-(2-(1H-pyrazol-1-yl)phenyl)-9-oxo-9H-fluorene-1-carboxamide

The title compound was prepared in a manner similar to example 20b. From N-(2-(1H-pyrazol-1-yl)phenyl)-7-nitro-9-oxo-9H-fluorene-1-carboxamide (0.100 g, 0.244 mmol) and Pd/C (5%, 0.067 g) under H_{2(g)} (45 psi) was obtained 0.013 g (14%) of the title compound as a brown solid: ¹H-NMR (DMSO-d₆): 10.71 (s, 1H), 8.24 (d, J=2.5 Hz, 1H), 8.16 (d, J=8.2 Hz, 1H), 7.68 (s, 1H), 7.63-7.38 (m, 6H), 7.22 (d, J=7.2 Hz, 1H), 6.75 (d, J=1.9 Hz, 1H), 6.70 (dd, J=8.0 and 1.9 Hz, 1H), 6.50 (s, 1H), 5.74 (br s, 2H).

### EXAMPLE 31

### 4,7-Dinitro-N-(2-(1H-pyrazol-1-yl)phenyl)-9-oxo-9H-fluorene-1-carboxamide

(a) Methyl 7-nitro-9-oxo-9H-fluorene-1-carboxylate. To an oven-dried 1-neck 200 mL reaction flask charged with a magnetic stir bar, under argon, fitted with a reflux condenser, at rt was added 7-nitro-9-oxo-9H-fluorene-1-carboxylic acid (7.60 g, 28.2 mmol) and MeOH (8.9 mL). To the yellow suspension was added concentrated HCl (˜1 mL). The yellow suspension was heated at reflux for 7 h and then cooled to rt. The yellow precipitate was filtered, washed with ether (100 mL) and collected on a buchner funnel. Purification by flash column chromatography (silica gel, elution with EtOAc:Hexanes, 1:2) gave 1.62 g (20%) of the title compound as a yellow solid: ¹H-NMR (CDCl₃): 8.47-8.42 (m, 2H), 7.81-7.63 (m, 4H), 4.03 (s, 3H).

(b) 4,7-Dinitro-9-oxo-9H-fluorene-1-carboxylic acid methyl ester. The title compound was prepared in a manner similar to example 29a. From H₂SO₄ (0.80 mL), HNO₃ (0.80 mL) and methyl 7-nitro-9-oxo-9H-fluorene-1-carboxylate (0.200 g, 0.706 mmol) was obtained 0.225 g (97%) of the title compound as a yellow solid: ¹H-NMR (DMSO-d₆): 8.67-8.60 (m, 2H), 8.44-8.33 (m, 3H), 4.00 (s, 3H).

(c) 4,7-Dinitro-9-oxo-9H-fluorene-1-carboxylic acid. To an oven-dried 1-neck 25 mL reaction flask charged with a magnetic stir bar, under argon, at rt was added 4,7-dinitro-9-oxo-9H-fluorene-1-carboxylic acid methyl ester (0.209 g, 0.636 mmol) and THF:H₂O (4:1, 6.85 mL) forming a yellow suspension. To the yellow suspension was added 1M NaOH (0.64 mL, 0.64 mmol) forming a black solution. The black solution was stirred at rt for 2 h, then the reaction solution was filtered through an amberlite resin column [Amberlite IR-120(plus) ion-exchange resin, 1.9 meq/mL, 2.00 g resin, prewashed with H₂O and 4:1, THF:H₂O] using 4:1, THF:H₂O as the eluant and collected the acidic filtrate (pH=3 to pH=5). The filtrate was concentrated to yield 0.166 g (83%) of the title compound as a yellow solid: ¹H-NMR (DMSO-d₆): 8.58-8.54 (m, 1H), 8.45-8.43 (m, 1H), 8.28-8.17 (m, 3H).

(d) 4,7-Dinitro-9-oxo-9H-fluorene-1-carbonyl chloride. The title compound was prepared in a manner similar to example 1a. From 4,7-dinitro-9-oxo-9H-fluorene-1-carboxylic acid (0.166 g, 0.528 mmol), oxalyl chloride (2.0M in dichloromethane, 0.36 mL, 0.71 mmol) and DMF (0.025 mL) was obtained 0.175 g (99%) of the title compound as a yellow solid.

(e) 4,7-Dinitro-N-(2-(1H-pyrazol-1-yl)phenyl)-9-oxo-9H-fluorene-1-carboxamide. The title compound was prepared in a manner similar to example 9. From 2-(1H-pyrazol-1-yl)aniline (0.080 g, 0.53 mmol) and 4,7-dinitro-9-oxo-9H-fluorene-1-carbonyl chloride (0.175 g, 0.529 mmol) was obtained 0.004 g (2%) of the title compound as a yellow solid: ¹H-NMR (CDCl₃): 10.83 (br s, 1H), 8.58-8.48 (m, 4H), 7.93-7.89 (m, 3H), 7.51-7.27 (m, 5H).

### EXAMPLE 32

### N-(2-(1H-Pyrazol-1-yl)phenyl)-dibenzothiophene-1-carboxamide

(a) Dibenzothiophene-1-carboxylic acid. The title compound was prepared in a manner similar to example 23a. From dibenzothiophene (1.00 g, 5.43 mmol) and nBuLi (1.6M in hexanes, 6.80 mL, 10.8 mmol) was obtained 0.625 g (50%) of the title compound as a brown solid: ¹H-NMR (DMSO-d₆): 8.75 (d, J=8.0 Hz, 1H), 8.54-8.51 (m, 1H), 8.28 (d, J=7.4 Hz, 1H), 8.18-8.15 (m, 1H), 7.76 (t, J=7.7 Hz, 1H), 7.68-7.61 (m, 2H).

(b) Dibenzothiophene-1-carbonyl chloride. To an oven-dried 1-neck 25 mL reaction flask charged with a magnetic stir bar, under argon, fitted with a reflux condenser, at rt was added dibenzothiophene-1-carboxylic acid (0.625 g, 2.74 mmol) and thionyl chloride (5.0 mL). The brown suspension was brought to reflux by heating to 90° C. and became a brown solution. After 45 minutes the solution was cooled to rt. The solvent was removed under vacuum at 70° C. for 1 h to yield 0.200 g (30%) of the title compound as a brown solid: ¹H-NMR (CDCl₃): 8.52-8.47 (m, 2H), 8.23-8.20 (m, 1H), 7.97-7.93 (m, 1H), 7.66 (t, J=7.8 Hz, 1H), 7.58-7.52 (m, 2H).

(c) N-(2-(1H-Pyrazol-1-yl)phenyl)-dibenzothiophene-1-carboxamide. The title compound was prepared in a manner to example 9. From 2-(1H-pyrazol-1-yl)aniline (0.122 g, 0.811 mmol) and dibenzothiophene-1-carbonyl chloride (0.200 g, 0.811 mmol) was obtained 0.045 g (15%) of the title compound as a white solid: 1H-NMR (CDCl₃): 11.72 (br s, 1H), 8.80 (d, J=8.2 Hz, 1H), 8.36 (d, J=8.0 Hz, 1H), 8.21-8.18 (m, 1H), 7.99 (d, J=7.7 Hz, 1H), 7.95-7.89 (m, 3H), 7.61 (t, J=7.7 Hz, 1H), 7.53-7.40 (m, 4H), 7.24-7.21 (m, 1H), 6.57 (t, J=2.2 Hz, 1H).

### EXAMPLE 33

### 9,9-Dioxo-N-(2-(1H-pyrazol-1-yl)phenyl)-dibenzothiophene-1-carboxamide

To an oven-dried 1-neck 10 mL reaction flask charged with a magnetic stir bar, under argon, at rt was added N-(2-(1H-pyrazol-1-yl)phenyl)-dibenzothiophene-1-carboxamide (0.015 g, 0.041 mmol) and anhydrous dichloromethane (0.41 mL). The yellow solution was stirred at rt for 5 minutes and then mCPBA (0.014 g, 0.081 mmol) was added. The yellow solution was stirred for 4 h, diluted with 10% K₂CO_{3(aq)} (10 mL), and extracted with EtOAc (50 mL). The organic layer was dried over MgSO₄, filtered and concentrated to give 0.055 g of a crude yellow solid. Purification by flash column chromatography (silica gel, gradient elution with EtOAc:Hexanes, 1:1; MeOH, 100%) gave 0.04 g (25%) of the title compound as a white solid: ¹H-NMR (CDCl₃): 11.61 (s, 1H), 8.84-8.80 (m, 1H), 7.99 (d, J=7.70 Hz, 1H), 7.91-7.87 (m, 3H), 7.82-7.75 (m, 3H), 7.66-7.57 (m, 2H), 7.42-7.38 (m, 2H), 7.26-7.24 (m, 1H), 6.52 (t, J=2.20 Hz, 1H); Mass Spectra (m/z) 424 (MNa⁺).

### EXAMPLE 34

### 9-Oxo-N-(2-(1H-pyrazol-1-yl)phenyl)-dibenzothiophene-1-carboxamide

To an oven-dried 1-neck 10 mL reaction flask charged with a magnetic stir bar, under argon, at rt was added N-(2-(1H-pyrazol-1-yl)phenyl)-dibenzothiophene-1-carboxamide (0.015 g, 0.041 mmol) and anhydrous dichloromethane (0.41 mL). The yellow solution was stirred at rt for 5 minutes and then mCPBA (0.014 g, 0.081 mmol) was added. The yellow solution was stirred for 4 h, diluted with 10% K₂CO_{3(aq)} (10 mL), and extracted with EtOAc (50 mL). The organic layer was dried over MgSO₄, filtered and concentrated to give 0.055 g of a crude yellow solid. Purification by flash column chromatography (silica gel, gradient elution with EtOAc:Hexanes, 1:1; MeOH, 100%) gave 0.05 g (31%) of the title compound as a white solid: ¹H-NMR (CDCl₃): 11.70 (s, 1H), 8.80 (d, J=8.2 Hz, 1H), 8.03-7.99 (m, 2H), 7.90-7.81 (m, 4H), 7.75 (t, J=7.7 Hz, 1H), 7.62-7.53 (m, 2H), 7.46-7.39 (m, 2H), 7.27-7.25 (m, 1H), 6.53 (t, J=2.2 Hz, 1H); Mass Spectra (m/z) 386 (MH⁺), 408 (MNa⁺).

### EXAMPLE 35

### N-(2-Hydroxy-phenyl)-9-oxo-9H-fluorene-1-carboxamide

To an oven-dried 1-neck 25 μL reaction flask charged with a magnetic stir bar, under argon, fitted with a reflux condenser, at rt was added 2-aminophenol (0.250 g, 2.29 mmol) and anhydrous pyridine (5.8 mL). The yellow solution was stirred for 5 minutes and then 9-oxo-9H-fluorene-1-carbonyl chloride (0.556 g, 2.29 μmol) was added forming a brown solution. The solution was heated at 60° C. for 1 h, cooled to rt, diluted with H₂O (2 mL) and then the solvent was evaporated. The resulting brown residue was diluted with H₂O (40 mL), extracted with EtOAc (250 mL), washed with H₂O (2×40 mL), 1M HCl (2×30 mL), H₂O (15 mL), brine (25 mL), dried over Na₂SO₄, filtered and concentrated to yield a brown solid. Purification by flash column chromatography (silica gel, elution EtOAc:Hexanes, 1:1) gave 0.147 g of a white solid. Recrystallization of the white solid with EtOH (5 mL) yielded 0.037 g (5%) of the title compound as a white solid: ¹H-NMR (CDCl₃): 12.74 (br s, 1H), 9.47 (s, 1H), 8.34 (d, J=7.7 Hz, 1H), 7.74-7.65 (m, 3H), 7.60-7.54 (m, 2H), 7.38-7.35 (m, 2H), 7.22 (t, J=7.9 Hz, 1H), 7.10 (d, J=7.7 Hz, 1H), 6.97 (t, J=7.5 Hz, 1H).

### EXAMPLE 36

### 9-Hydroxy-N-(2-methylphenyl)-9H-fluorene-1-carboxamide

9-Oxo-N-(2-methylphenyl)-9H-fluorene-1-carboxamide (28 mg, 0.0893 mmol) was suspended in 5 mL of methanol at 0° C. and sodium borohydride was added in two portions (15 mg×2, 0.40 mmol). The reaction mixture was warmed to room temperature and continued stirring for 1 h. The reaction mixture was diluted with 25 mL of ethyl acetate and extracted with 1N HCl (25 mL), water and saturated sodium chloride respectively. The organic layer was dried over anhydrous sodium sulfate, filtered and concentrated. The crude product was purified by chromatography to obtain the title compound (12 mg, 0.038 mmol, 43%). ¹H NMR (CDCl₃, 300 MHz): δ 8.64 (s, 1H, broad), 7.82-7.85 (m, 2H), 7.66-7.72 (m, 3H), 7.51 (t, 7.5, 1H), 7.31-7.20 (m, 1H), 5.83 (d, 4.2, 1H), 5.15 (d, 4.5, 1H, D₂O exchangeable), 2.36 (s, 3H).

### EXAMPLE 37

### 7-Azido-N-(2-(1H-pyrazol-1-yl)phenyl)-9-oxo-9H-fluorene-1-carboxamide

A solution of sodium nitrite (3 mg, 0.043 mmol) in 250 uL of water was added drop wise to a solution of 7-amino-N-(2-(1H-pyrazol-1-yl)phenyl)-9-oxo-9H-fluorene-1-carboxamide (5.1 mg, 0.0134 mmol) in 1 mL of 2N HCl at 0° C. The mixture was stirred at the same temperature for 15 min and a solution of sodium azide (3 mg, 0.0461 mmol) in 250 uL of water was added and the mixture was stirred for 1 h at the same temperature. The reaction mixture was diluted with 20 mL ethyl acetate and washed with saturated sodium bicarbonate, the organic layer was dried over anhydrous sodium sulfate, filtered and concentrated. The residue was purified by chromatography (30% ethyl acetate/hexane) to obtain the title compound (3.9 mg, 0.0095 mmol, 72%). ¹H NMR (CDCl₃, 300 MHz): δ 11.09 (s, 1H, broad), 8.55 (d, J=8.1, 1H), 7.83 (m, 1H), 7.45-7.77 (m, 1H), 7.39-7.60 (m, 6H), 7.26-7.29 (m, 2H), 7.12-7.15 (m, 1H), 6.43 (s, 1H).

### EXAMPLE 38

### N-(2-(4-Bromo-1H-pyrazol-1-yl)phenyl)-7-nitro-9-oxo-9H-fluorene-1-carboxamide

A solution of bromine (60 mg, 0.375 mmol) in 1 mL of glacial acetic acid was added drop wise to a solution of N-(2-(1H-pyrazol-1-yl)phenyl)-7-nitro-9-oxo-9H-fluorene-1-carboxamide (48 mg, 0.117 mmol) in a mixture of 3 mL of glacial acetic acid and 2 mL of dichloromethane at 0° C. The mixture was stirred at 0° C. for 30 min and warmed to room temperature, more bromine (30 mg, 0.188 mmol) in 1 mL of glacial acetic acid was added and mixture was heated at 50° C. for 45 min. The contents of the reaction mixture was evaporated under reduced pressure and the residue was dissolved in ethyl acetate, washed with saturated bicarbonate and the organic layer was dried over anhydrous sodium sulfate, filtered and concentrated. The residue was purified by chromatography (35% ethyl acetate/hexane) to obtain the title compound (39 mg, 0.080 mmol, 68%). ¹H NMR (DMSO, 300 MHz): δ 10.41 (s, 1H), 8.54-8.57 (m, 1H), 8.45 (s, 1H), 8.24 (d, J=1.8, 1H), 8.18 (d, J=8.4, 1H), 8.17 (d, J=7.5, 1H), 8.0 (d, J=7.8, 1H), 7.82-7.86 (m, 2H), 7.53-7.60 (m, 3H), 7.42 (t, J=7.8, 1H).

### EXAMPLE 39

### Identification of N-(2-Methylphenyl)-9-oxo-9H-fluorene-1-carboxamide and other Analogs as Antineoplastic Compounds that are Caspase Cascade Activators and Apoptosis Inducers

Human breast cancer cell lines T-47D was grown according to media component mixtures designated by American Type Culture Collection+10% FCS (Invitrogen Corporation), in a 5% CO₂-95% humidity incubator at 37° C. T-47D and ZR-75-1 cells were maintained at a cell density between 30 and 80% confluency and for HL-60 at a cell density of 0.1 to 0.6×10⁶ cells/mL. Cells were harvested at 600×g and resuspended at 0.65×10⁶ cells/mL into appropriate media+10% FCS. An aliquot of 45 μL of cells was added to a well of a 96-well microtiter plate containing 5 μL of a 10% DMSO in RPMI-1640 media solution containing 1.6 to 100 μM of N-(2-methylphenyl)-9-oxo-9H-fluorene-1-carboxamide or other test compound (0.16 to 10 μM final). An aliquot of 45 μL of cells was added to a well of a 96-well microtiter plate containing 5 μL of a 10% DMSO in RPMI-1640 media solution without test compound as the control sample. The samples were mixed by agitation and then incubated at 37° C. for 24 h in a 5% CO₂-95% humidity incubator. After incubation, the samples were removed from the incubator and 50 μL of a solution containing 20 μM of N—(Ac-DEVD)-N′-ethoxycarbonyl-R110 fluorogenic substrate (SEQ ID NO:1) (Cytovia, Inc.; U.S. Pat. No. 6,335,429), 20% sucrose (Sigma), 20 mM DTT (Sigma), 200 mM NaCl (Sigma), 40 mM Na PIPES buffer pH 7.2 (Sigma), and 500 μg/mL lysolecithin (Calbiochem) was added. The samples were mixed by agitation and incubated at room temperature. Using a fluorescent plate reader (Model 1420 Wallac Instruments), an initial reading (T=0) was made about 1-2 min after addition of the substrate solution, employing excitation at 485 nm and emission at 530 nm, to determine the background fluorescence of the control sample. After about 3 h of incubation, the samples were read for fluorescence as above (T=3 h).

Calculation:

The Relative Fluorescence Unit values (RFU) were used to calculate the sample readings as follows:
RFU_{(T=3h)}−Control RFU_{(T=0)}=Net RFU_{(T=3h)}

The activity of caspase cascade activation was determined by the ratio of the net RFU value for N-(2-methylphenyl)-9-oxo-9H-fluorene-1-carboxamide and other test compounds to that of control samples. The EC₅₀ (nM) was determined by a sigmoidal dose-response calculation (Prism 2.0, GraphPad Software Inc.). The caspase activity (Ratio) and potency (EC₅₀) are summarized in Table I:

1.

TABLE I
Caspase Activity and Potency

| | T-47D | |
|---|---|---|
| | Ratio EC₅₀ | |
| The Compound or Example # | (nM) | |
| N-(1-Naphthalen-1-yl)-9-oxo-9H-fluorene-1- | 3.2 | 6000 |
| carboxamide | | |
| N-(2-Methylphenyl)-9-oxo-9H-fluorene-1- | 11.2 | 570 |
| carboxamide (Example A) | | |
| N-(3-Methylphenyl)-9-oxo-9H-fluorene-1- | 1.3 | >10000 |
| carboxamide | | |
| N-(4-Methylphenyl)-9-oxo-9H-fluorene-1- | 1.4 | >10000 |
| carboxamide | | |
| N-(4-Methoxyphenyl)-9-oxo-9H-fluorene-1- | 1.8 | >10000 |
| carboxamide | | |
| N-(2-Phenylphenyl)-9-oxo-9H-fluorene-1- | 8.1 | 1500 |
| carboxamide | | |
| N-(2-Difluoromethoxyphenyl)-9-oxo-9H-fluorene-1- | 5.9 | 688 |
| carboxamide | | |
| N-(2-(Methoxycarbonyl)phenyl)-9-oxo-9H-fluorene- | 7.1 | 419 |
| 1-carboxamide | | |
| N-(2-Cholorophenyl)-9-oxo-9H-fluorene-1- | 7.4 | 1055 |
| carboxamide | | |
| N-(2-Fluorophenyl)-9-oxo-9H-fluorene-1- | 5.7 | 1957 |
| carboxamide | | |
| N-(2-Cyanophenyl)-9-oxo-9H-fluorene-1- | 2.3 | 2000 |
| carboxamide | | |
| N-(2-Bromophenyl)-9-oxo-9H-fluorene-1- | 9.1 | 3522 |
| carboxamide | | |
| N-(2-Ethoxyphenyl)-9-oxo-9H-fluorene-1- | 8.2 | 2071 |
| carboxamide | | |
| 1 | 3.1 | 5675 |
| 2 | 4.2 | 2234 |
| 3 | 7.4 | 1922 |
| 4 | 1.4 | >10000 |
| 5 | 1.2 | >10000 |
| 6 | 1.9 | >10000 |
| 7 | 1.4 | >10000 |
| 8 | 1.1 | >10000 |
| 9 | 7.7 | 979 |
| 10 | 8.0 | 1539 |
| 11 | 1.2 | >10000 |
| 12 | 8.9 | 1580 |
| 13 | 6.0 | 1095 |
| 14 | 5.7 | 2883 |
| 15 | 6.6 | 577 |
| 16 | 0.8 | >10000 |
| 17 | 1.4 | >10000 |
| 18 | 1.8 | >10000 |
| 19 | 0.9 | >10000 |
| 20 | 6.7 | 697 |
| 21 | 0.8 | >10000 |
| 22 | 1.0 | >10000 |
| 23 | 1.1 | >10000 |
| 24 | 1.8 | >10000 |
| 25 | 2.2 | 2513 |
| 26 | 1.2 | >10000 |
| 27 | 4.0 | 1400 |
| 28 | 1.2 | >10000 |
| 29 | 5.3 | 231 |
| 30 | 4.4 | 881 |
| 31 | 2.3 | 6051 |
| 32 | 2.7 | 1277 |
| 33 | 1.6 | >10000 |
| 34 | 1.2 | >10000 |
| 35 | 1.3 | >10000 |
| 36 | 3.3 | 3701 |
| 37 | 3.0 | 681 |
| 38 | 8.0 | 302 |

Thus, N-(2-methylphenyl)-9-oxo-9H-fluorene-1-carboxamide (Example A) and other analogs are identified as potent caspase cascade activators and antineoplastic compounds in this assay.

### EXAMPLE 40

### Identification of N-(2-Methylphenyl)-9-oxo-9H-fluorene-1-carboxamide as an Antineoplastic Compound that Inhibits Cell Proliferation (GI₅₀)

T-47D and MX1 cells were grown and harvested as in Example 39. An aliquot of 90 μL of cells (2.2×10⁴ cells/mL) was added to a well of a 96-well microtiter plate containing 10 μl of a 10% DMSO in RPMI-1640 media solution containing 1 nM to 100 μM of N-(2-methylphenyl)-9-oxo-9H-fluorene-1-carboxamide (0.1 nM to 10 μM final) or other test compound. An aliquot of 90 μL of cells was added to a well of a 96-well microtiter plate containing 10 μL of a 10% DMSO in RPMI-1640 media solution without compound as the control sample for maximal cell proliferation (A_{Max}). The samples were mixed by agitation and then incubated at 37° C. for 48 h in a 5% CO₂-95% humidity incubator. After incubation, the samples were removed from the incubator and 20 μL of CellTiter 96 AQ_{UEOUS} One Solution Cell Proliferation™ reagent (Promega) was added. The samples were mixed by agitation and incubated at 37° C. for 2-4 h in a 5% CO₂-95% humidity incubator. Using an absorbance plate reader (Model 1420 Wallac Instruments), an initial reading (T=0) was made about 1-2 min after addition of the solution, employing absorbance at 490 nm. This determines the possible background absorbance of the test compounds. No absorbance for N-(2-methylphenyl)-9-oxo-9H-fluorene-1-carboxamide was found at 490 nm. After the 2-4 h incubation, the samples were read for absorbance as above (A_{Test}).

Baseline for GI₅₀ (dose for 50% inhibition of cell proliferation) of initial cell numbers were determined by adding an aliquot of 90 μL of cells or 90 μL of media, respectively, to wells of a 96-well microtiter plate containing 10 μL of a 10% DMSO in RPMI-1640 media solution. The samples were mixed by agitation and then incubated at 37° C. for 0.5 h in a 5% CO₂-95% humidity incubator. After incubation, the samples were removed from the incubator and 20 μL of CellTiter 96 AQ_{UEOUS} One Solution Cell Proliferation™ reagent (Promega) was added. The samples were mixed by agitation and incubated at 37° C. for 2-4 h in a 5% CO₂-95% humidity incubator. Absorbance was read as above, (A_{Start}) defining absorbance for initial cell number used as baseline in GI₅₀ determinations.

Calculation:

GI₅₀ (dose for 50% inhibition of cell proliferation) is the concentration where [(A_{Test}−Aₛₜₐᵣₜ)/(A_{Max}−Aₛₜₐᵣₜ)]=0.5.

The GI₅₀ (nM) are summarized in Table II:

2.

TABLE II
GI₅₀ in Cancer Cells

| | |
|---|---|
| The Compound | GI₅₀ (nM) |

| of Example # | T-47D | MX1 |
|---|---|---|
| Example A | 146 | 549 |
| Example 9 | 451 | 1000 |
| Example 10 | 665 | 736 |
| Example 13 | 5042 | 6000 |
| Example 15 | 600 | 2000 |
| Example 20 | 233 | 8000 |
| Example 27 | 45 | 90 |
| Example 28 | >10000 | >10000 |
| Example 29 | 166 | 150 |
| Example 32 | 2474 | 4346 |
| Example 32 | 2474 | 4346 |
| Example 37 | 415 | 200 |
| Example 38 | 429 | 315 |

Thus, N-(2-methylphenyl)-9-oxo-9H-fluorene-1-carboxamide (Example A) and analogs are identified as antineoplastic compound that inhibits cell proliferation.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating or ameliorating a disorder responsive to the induction of apoptosis in an animal suffering therefrom, comprising administering to an animal in need of such treatment an effective amount of a compound of Formula I: 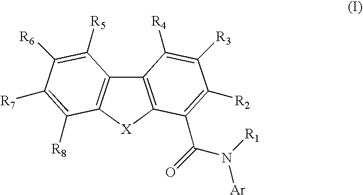
or a pharmaceutically acceptable salt or prodrug thereof, wherein:
X is CR₉R₁₀, O, NR₉, S, C═O, SO, or SO₂;
Ar is optionally substituted and is aryl, heteroaryl, saturated carbocyclic, partially saturated carbocylic, saturated heterocyclic, partially saturated heterocyclic, arylalkyl, or heteroarylalkyl;
R₁ is hydrogen or optionally substituted C₁₋₁₀ alkyl;
R₂-R₈ are independently hydrogen, halo, haloalkyl, aryl, optionally substituted fused aryl, optionally substituted fused heteroaryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate; and
R₉ and R₁₀ are independently hydrogen, hydroxy or optionally substituted C₁₋₁₀ alkyl.

2. The method of claim 1, wherein Ar is an optionally substituted phenyl or pyridyl.

3. The method of claim 1, wherein Ar is an optionally substituted phenyl.

4. The method of claim 1, wherein said compound is selected from the group consisting of:
N-(1-Naphthalen-1-yl)-9-oxo-9H-fluorene-1-carboxamide;
N-(2-Methylphenyl)-9-oxo-9H-fluorene-1-carboxamide;
N-(2-Phenylphenyl)-9-oxo-9H-fluorene-1-carboxamide;
N-(2-Difluoromethoxyphenyl)-9-oxo-9H-fluorene-1-carboxamide;
N-(2-(Methoxycarbonyl)phenyl)-9-oxo-9H-fluorene-1-carboxamide;
N-(2-Cholorophenyl)-9-oxo-9H-fluorene-1-carboxamide;
N-(2-Fluorophenyl)-9-oxo-9H-fluorene-1-carboxamide;
N-(2-Cyanophenyl)-9-oxo-9H-fluorene-1-carboxamide;
N-(2-Bromophenyl)-9-oxo-9H-fluorene-1-carboxamide;
N-(2-Ethoxyphenyl)-9-oxo-9H-fluorene-1-carboxamide;
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide;
N-(3-Methylpyridin-2-yl)-9-Oxo-9H-fluorene-1-carboxamide; and
N-(2-(1H-Pyrazol-1-yl)-phenyl)-9H-fluorene-1-carboxamide.
or a pharmaceutically acceptable salt or prodrug thereof.

5. The method of claim 1, wherein said compound is selected from the group consisting of:
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
N-(2-(1H-Pyrazol-1-yl)phenyl)-dibenzothiophene-1-carboxamide;
9-Hydroxy-N-(2-methylphenyl)-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide;
or a pharmaceutically acceptable salt or prodrug thereof.

6. The method of claim 1, wherein said compound has the Formula II: 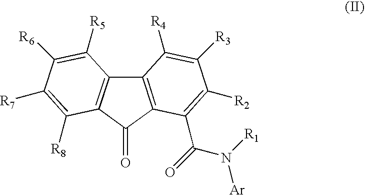
or a pharmaceutically acceptable salt or prodrug thereof.

7. The method of claim 6, wherein Ar is an optionally substituted phenyl.

8. The method of claim 6, wherein said compound is selected from the group consisting of:
N-(1-Naphthalen-1-yl)-9-oxo-9H-fluorene-1-carboxamide;
N-(2-Methylphenyl)-9-oxo-9H-fluorene-1-carboxamide;
N-(2-Phenylphenyl)-9-oxo-9H-fluorene-1-carboxamide;
N-(2-Difluoromethoxyphenyl)-9-oxo-9H-fluorene-1-carboxamide;
N-(2-(Methoxycarbonyl)phenyl)-9-oxo-9H-fluorene-1-carboxamide;
N-(2-Cholorophenyl)-9-oxo-9H-fluorene-1-carboxamide;
N-(2-Fluorophenyl)-9-oxo-9H-fluorene-1-carboxamide;
N-(2-Cyanophenyl)-9-oxo-9H-fluorene-1-carboxamide;
N-(2-Bromophenyl)-9-oxo-9H-fluorene-1-carboxamide;
N-(2-Ethoxyphenyl)-9-oxo-9H-fluorene-1-carboxamide;
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide; and
N-(3-Methylpyridin-2-yl)-9-Oxo-9H-fluorene-1-carboxamide;
or a pharmaceutically acceptable salt or prodrug thereof.

9. The method of claim 6, wherein said compound is selected from the group consisting of:
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide;
or a pharmaceutically acceptable salt or prodrug thereof.

10. The method of claim 1, wherein said compound has the Formula III: 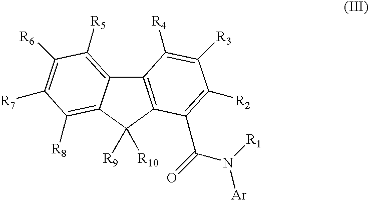
or a pharmaceutically acceptable salt or prodrug thereof.

11. The method of claim 10, wherein Ar is an optionally substituted phenyl.

12. The method of claim 10, wherein R₉ and R₁₀ are hydrogen.

13. The method of claim 10, wherein said compound is
N-(2-(1H-Pyrazol-1-yl)-phenyl)-9H-fluorene-1-carboxamide; and
9-Hydroxy-N-(2-methylphenyl)-9H-fluorene-1-carboxamide;
or a pharmaceutically acceptable salt or prodrug thereof.

14. The method of claim 1, wherein said compound has the Formula IV: 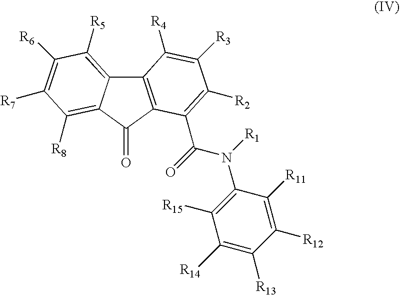
or a pharmaceutically acceptable salt or prodrug thereof, wherein:
R₁₁-R₁₅ are independently hydrogen, halo, haloalkyl, aryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate.

15. The method of claim 14, wherein one of the R₁₁ or R₁₅ is not hydrogen.

16. The method of claim 14, wherein said compound is selected from the group consisting of:
N-(2-Methylphenyl)-9-oxo-9H-fluorene-1-carboxamide;
N-(2-Phenylphenyl)-9-oxo-9H-fluorene-1-carboxamide;
N-(2-Difluoromethoxyphenyl)-9-oxo-9H-fluorene-1-carboxamide;
N-(2-(Methoxycarbonyl)phenyl)-9-oxo-9H-fluorene-1-carboxamide;
N-(2-Cholorophenyl)-9-oxo-9H-fluorene-1-carboxamide;
N-(2-Fluorophenyl)-9-oxo-9H-fluorene-1-carboxamide;
N-(2-Cyanophenyl)-9-oxo-9H-fluorene-1-carboxamide;
N-(2-Bromophenyl)-9-oxo-9H-fluorene-1-carboxamide;
N-(2-Ethoxyphenyl)-9-oxo-9H-fluorene-1-carboxamide;
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide; and
N-(2-Dimethylaminophenyl)-9-oxo-9H-fluorene-1-carboxamide;
or a pharmaceutically acceptable salt or prodrug thereof.

17. The method of claim 14, wherein said compound is selected from the group consisting of:
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide;
or a pharmaceutically acceptable salt or prodrug thereof.

18. A method for treating or ameliorating cancer, comprising administering to an animal in need of such treatment an effective amount of a compound of Formula I: 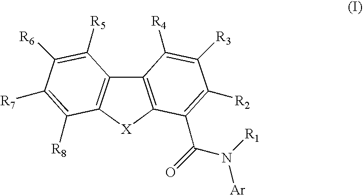
and pharmaceutically acceptable salts and prodrugs thereof, wherein:
X is CR₉R₁₀, O, NR₉, S, C═O, SO, or SO₂;
Ar is optionally substituted and is aryl, heteroaryl, saturated carbocyclic, partially saturated carbocylic, saturated heterocyclic, partially saturated heterocyclic, arylalkyl, or heteroarylalkyl;
R₁ is hydrogen or optionally substituted C₁₋₁₀ alkyl;
R₂-R₈ are independently hydrogen, halo, haloalkyl, aryl, optionally substituted fused aryl, optionally substituted fused heteroaryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate; and
R₉ and R₁₀ are independently hydrogen, hydroxy or optionally substituted C₁₋₁₀ alkyl.

19. The method of claim 18, wherein said animal is a mammal.

20. The method of claim 18, wherein said cancer is selected from the group consisting of Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinomas, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma.

21. A method for the treatment or amelioration of drug-resistant cancer, comprising administering to an animal in need of such treatment or amelioration an effective amount of a compound of the Formula I: 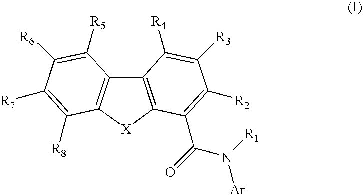
and pharmaceutically acceptable salts and prodrugs thereof, wherein:
X is CR₉R₁₀, O, NR₉, S, C═O, SO, or SO₂;
Ar is optionally substituted and is aryl, heteroaryl, saturated carbocyclic, partially saturated carbocylic, saturated heterocyclic, partially saturated heterocyclic, arylalkyl, or heteroarylalkyl;
R₁ is hydrogen or optionally substituted C₁₋₁₀ alkyl;
R₂-R₈ are independently hydrogen, halo, haloalkyl, aryl, optionally substituted fused aryl, optionally substituted fused heteroaryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate; and
R₉ and R₁₀ are independently hydrogen, hydroxy or optionally substituted C₁₋₁₀ alkyl.

22. The method of claim 21, wherein said animal is a mammal.

23. The method of claim 18 or 21, additionally comprising administering at least one known cancer chemotherapeutic agent, or a pharmaceutically acceptable salt of said agent.

24. The method of claim 18 or 21, wherein said compound is administered together with at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylomithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

25. The method of claim 18 or 21, additionally comprising treating said animal with radiation-therapy.

26. The method of claim 1, wherein said disorder is rheumatoid arthritis.

27. The method of claim 1, wherein said disorder is inflammation.

28. The method of claim 1, wherein said disorder is inflamatory bowel disease.

29. The method of claim 1, wherein said disorder is Crohn's disease.

30. The method of claim 1, wherein said disorder is ulcerative colitis.

31. The method of claim 1, wherein said disorder is a skin disease.

32. The method of claim 31, wherein said disorder is psoriasis.

33. The method according to claim 1, wherein said disorder is an infectious viral disease.

34. A pharmaceutical composition comprising a pharmaceutically acceptable carrier and a compound of Formula I: 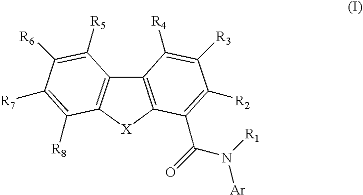
and pharmaceutically acceptable salts and prodrugs thereof, wherein:
X is CR₉R₁₀, O, NR₉, S, C═O, SO, or SO₂;
Ar is optionally substituted and is aryl, heteroaryl, saturated carbocyclic, partially saturated carbocylic, saturated heterocyclic, partially saturated heterocyclic, arylalkyl, or heteroarylalkyl;
R₁ is hydrogen or optionally substituted C₁₋₁₀ alkyl;
R₂-R₈ are independently hydrogen, halo, haloalkyl, aryl, optionally substituted fused aryl, optionally substituted fused heteroaryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate; and
R₉ and R₁₀ are independently hydrogen, hydroxy or optionally substituted C₁₋₁₀ alkyl.

35. The pharmaceutical composition of claim 34, wherein said compound is selected from the group consisting of:
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide;
N-(3-Methylpyridin-2-yl)-9-Oxo-9H-fluorene-1-carboxamide; and
N-(2-(1H-Pyrazol-1-yl)-phenyl)-9H-fluorene-1-carboxamide; or a pharmaceutically acceptable salt or prodrug thereof.

36. The pharmaceutical composition of claim 34, wherein said compound is selected from the group consisting of:
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
N-(2-(1H-Pyrazol-1-yl)phenyl)-dibenzothiophene-1-carboxamide;
9-Hydroxy-N-(2-methylphenyl)-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide;
or a pharmaceutically acceptable salt or prodrug thereof.

37. The pharmaceutical composition of claim 35 and 36, additionally comprising at least one known cancer chemotherapeutic agent, or a pharmaceutically acceptable salt of said agent.

38. The pharmaceutical composition of claim 37, wherein said known cancer therapeutic agent is selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gamcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylomithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

39. A compound of Formula IV: 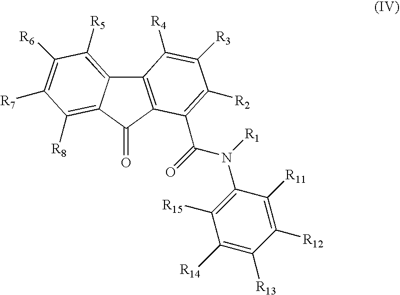
and pharmaceutically acceptable salts and prodrugs thereof, wherein:
R₁ is hydrogen or optionally substituted C₁₋₁₀ alkyl;
R₂-R₈ and R₁₁-R₁₅ are independently hydrogen, halo, haloalkyl, aryl, optionally substituted fused aryl, optionally substituted fused heteroaryl, carbocyclic, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido, alkylthiol, alkylsulfonyl or alkylcarboxylate;
with the proviso that when R₁₂-R₁₅ is hydrogen, then R₁₁ is other than hydrogen, Me, Ph, OCHF₂, CO₂Me, Cl, F, Br, CN, OMe, or OEt.

40. A compound selected from the group consisting of:
N-(2-Morpholinophenyl)-9-oxo-9H-fluorene-1-carboxamide;
N-(2-(1H-Pyrrolo-1-yl)-phenyl)-9-oxo-9H-fluorene-1-carboxamide;
9-Oxo-N-(2-(piperidin-1-yl)-phenyl)-9H-fluorene-1-carboxamide;
N-(2-(1H-Imidazol-1-yl)-phenyl)-9-oxo-9H-fluorene-1-carboxamide;
9-Oxo-N-(pyridin-2-yl)-9H-fluorene-1-carboxamide;
N-(2,6-Dimethylphenyl)-9-oxo-9H-fluorene-1-carboxamide;
N-(2-(1H-tetrazol-5-yl)phenyl)-9-oxo-9H-fluorene-1-carboxamide;
N-(2-(1H-Pyrazol-1-yl)-phenyl)-9-oxo-9H-fluorene-1-carboxamide;
N-(2-Dimethylaminophenyl)-9-oxo-9H-fluorene-1-carboxamide;
N-(3-Methylpyridin-2-yl)-9-Oxo-9H-fluorene-1-carboxamide; and
N-(2-(1H-Pyrazol-1-yl)-phenyl)-9H-fluorene-1-carboxamide;
or a pharmaceutically acceptable salt or prodrug thereof.

41. A compound selected from the group consisting of:
N-(2-Bromomethylphenyl)-9-oxo-9H-fluorene-1-carboxamide;
N-(2-Methoxyphenyl)-9-oxo-9H-fluorene-1-carboxamide;
N-(2-Dimethylaminomethylphenyl)-9-oxo-9H-fluorene-1-carboxamide;
N-(2-Nitrophenyl)-9-oxo-9H-fluorene-1-carboxamide;
N-(2-Azido-phenyl)-9-oxo-9H-fluorene-1-carboxamide;
7-Nitro-N-(2-(1H-pyrazol-1-yl)phenyl)-9-oxo-9H-fluorene-1-carboxamide;
7-Amino-N-(2-(1H-pyrazol-1-yl)phenyl)-9-oxo-9H-fluorene-1-carboxamide;
4,7-Dinitro-N-(2-(1H-pyrazol-1-yl)phenyl)-9-oxo-9H-fluorene-1-carboxamide;
N-(2-(1H-Pyrazol-1-yl)phenyl)-dibenzothiophene-1-carboxamide;
9-Hydroxy-N-(2-methylphenyl)-9H-fluorene-1-carboxamide;
7-Azido-N-(2-(1H-pyrazol-1-yl)phenyl)-9-oxo-9H-fluorene-1-carboxamide; and
N-(2-(4-Bromo-1H-pyrazol-1-yl)phenyl)-7-nitro-9-oxo-9H-fluorene-1-carboxamide;
or a pharmaceutically acceptable salt or prodrug thereof.
